(19) United States
(12) Patent Application Publication
(10) Pub. No.: US 2026/0035724 A1
(43) Pub. Date: Feb. 5, 2026

(54) SOLID STATE POLYNUCLEOTIDE ASSEMBLY

(71) Applicant: Switchback Systems, Inc., Santa Cruz, CA (US)

(72) Inventors: Mary S. NOÉ, Santa Cruz, CA (US); Alyssa K. BURKHARDT, Santa Cruz, CA (US); Marina L. RAMON, Santa Cruz, CA (US)

(73) Assignee: Switchback Systems, Inc., Santa Cruz, CA (US)

(21) Appl. No.: 19/071,747

(22) Filed: Mar. 5, 2025

Related U.S. Application Data
(63) Continuation of application No. PCT/US23/31977, filed on Sep. 5, 2023.
(60) Provisional application No. 63/404,109, filed on Sep. 6, 2022.

(51) Int. Cl. C12P 19/34

(52) U.S. Cl. CPC C12P 19/34

(57) ABSTRACT

Provided herein are immobilized polynucleotide templates and methods of producing immobilized polynucleotide templates. In some embodiments, the immobilized polynucleotide templates are single-stranded. In some embodiments, the immobilized polynucleotide templates are double-stranded. In embodiments, the immobilized polynucleotide templates are amplified to yield amplified double-stranded polynucleotides. Also provided herein are methods of producing error-corrected single-stranded and double-stranded polynucleotides.

### CONTINUING APPLICATION DATA

This application is a continuation of PCT/US23/31977, filed Sep. 5, 2023, which claims the benefit of U.S. Provisional No. 63/404,109, filed Sep. 6, 2022, the entire content of each of which is fully incorporated herein by reference.

### SEQUENCE LISTING

This application contains a Sequence Listing electronically submitted via Patent Center to the United States Patent and Trademark Office as an xml file entitled “69385-726.301_SL.xml” having a size of 68.7 kilobytes and created on Mar. 5, 2025. The information contained in the Sequence Listing is incorporated by reference herein.

### FIELD

The present disclosure relates to a method for solid-state synthesis of polynucleotides. The present disclosure additionally relates to templates for solid-state polynucleotide amplification and methods of making the same.

### SUMMARY

The present disclosure provides templates, methods, and kits for solid-state synthesis of polynucleotides. In contrast to current methods for solid-state synthesis of polynucleotides, the methods disclosed herein result in a more homogenous product and enable synthesis of longer polynucleotides.

In one aspect, the present disclosure provides a method for producing an immobilized double-stranded polynucleotide template including providing a solid support including an attached polynucleotide, wherein the attached polynucleotide is attached to the solid support. The method then includes annealing multiple polynucleotides to the attached polynucleotide to yield a double-stranded immobilized polynucleotide assembly comprising annealed polynucleotides. At this stage, the double-stranded immobilized polynucleotide assembly includes a top strand with a discontinuous phosphate backbone and a bottom strand with a discontinuous phosphate backbone, wherein the top strand is attached to the solid support. The method then includes treating the double-stranded immobilized polynucleotide assembly with a ligase to create a double-stranded immobilized polynucleotide, wherein the multiple polynucleotides of the top strand are ligated to result in the top strand including a continuous phosphate backbone and the multiple polynucleotides of the bottom strand are ligated to result in the bottom strand comprising a continuous phosphate backbone. The double-stranded immobilized polynucleotide is then treated with an enzyme to remove one or more errors present in the double-stranded immobilized polynucleotide to yield a double-stranded immobilized polynucleotide template.

In some embodiments, the method includes denaturing the double-stranded immobilized polynucleotide template and removing the bottom strand to yield an immobilized single-stranded polynucleotide template.

In some embodiments, the method further includes amplifying the double-stranded immobilized polynucleotide template or the immobilized single-stranded polynucleotide template enzymatically with two primers to result in an amplified template, wherein amplifying includes multiple rounds of amplification. In some embodiments, the method further includes treating the amplified template with 3′-5′ exonuclease and separating the amplified template from reaction components to yield an amplified double-stranded polynucleotide.

In another aspect, this disclosure describes a solid support including one or more of the immobilized single-stranded polynucleotide templates described herein.

In another aspect, this disclosure describes a kit including the solid support including one or more of the immobilized single-stranded polynucleotide templates described herein.

The above summary of the present disclosure is not intended to describe each disclosed embodiment or every implementation of the present disclosure. The description and figures that follow more particularly exemplify illustrative embodiments. In several places throughout the application, guidance is provided through lists of examples, which examples may be used in various combinations. In each instance, the recited list serves only as a representative group and should not be interpreted as an exclusive list. Thus, the scope of the present disclosure should not be limited to the specific illustrative structures described herein, but rather extends at least to the structures described by the language of the embodiments, and the equivalents of those structures. Any of the elements that are positively recited in this disclosure as alternatives may be explicitly included in the embodiments or excluded from the embodiments, in any combination as desired. Although various theories and possible mechanisms may have been discussed herein, in no event should such discussions serve to limit the claimable subject matter.

All scientific and technical terms used herein have meanings commonly used in the art unless otherwise specified. The definitions provided herein are to facilitate understanding of certain terms used frequently herein and are not meant to limit the scope of the present disclosure.

Herein, the term “comprises” and variations thereof do not have a limiting meaning where these terms appear in the description and embodiments. Such terms will be understood to imply the inclusion of a stated step or element or group of steps or elements but not the exclusion of any other step or element or group of steps or elements. By “consisting of is meant including, and limited to, whatever follows the phrase “consisting of” Thus, the phrase “consisting of indicates that the listed elements are required or mandatory, and that no other elements may be present. By “consisting essentially of is meant including any elements listed after the phrase and limited to other elements that do not interfere with or contribute to the activity or action specified in the disclosure for the listed elements. Thus, the phrase “consisting essentially of indicates that the listed elements are required or mandatory, but that other elements are optional and may or may not be present depending upon whether or not they materially affect the activity or action of the listed elements. Any of the elements or combinations of elements that are recited in this disclosure in open-ended language (e.g., comprise and derivatives thereof), are considered to additionally be recited in closed-ended language (e.g., consist and derivatives thereof) and in partially closed-ended language (e.g., consist essentially, and derivatives thereof).

In this application, terms such as “a,” “an,” and “the” are not intended to refer to only a singular entity but include the general class of which a specific example may be used for illustration. The terms “a,” “an,” and “the” are used interchangeably with the term “at least one” and “one or more” and include one, two, three, etc., including all of the items these terms modify. The phrases “at least one of and “comprises at least one of as well as “one or more” and “comprises one or more” followed by a list refers to any of the items in the list and any combination of two or more items in the list.

As used herein, the term “or” is generally employed in its usual sense including “and/or” unless the content clearly dictates otherwise. The term “and/or” means one or all the listed elements or a combination of any two or more of the listed elements.

Also herein, all numbers are assumed to be modified by the term “about” and in certain embodiments, preferably, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

Herein, “up to” a number (e.g., up to 50) includes the number (e.g., 50). The term “about” is used here in conjunction with numeric values to include normal variations in measurements as expected by persons skilled in the art and is understood have the same meaning as “approximately” and to cover a typical margin of error, such as ±5% of the stated value.

Also herein, the recitations of numerical ranges by endpoints include all numbers subsumed within that range as well as the endpoints (e.g., 1 to 5 includes 1, 1.5, 2, 2.75, 3, 3.80, 4, 5, etc.) and any sub-ranges (e.g., 1 to 5 includes 1 to 4, 1 to 3, 2 to 4, etc.).

The term “in the range” or “within a range” (and similar statements) includes the endpoints of the stated range.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

As used herein, “providing” in the context of a composition, an article, a solid support, or a polynucleotide means making the composition, article, or polynucleotide, purchasing the composition, article, a solid support, or polynucleotide, or otherwise obtaining the composition, article, or polynucleotide.

Unless otherwise indicated, the terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds, adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages or modified sugar residues, or non-canonical/chemically modified nucleobases and combinations thereof, which are synthetic, naturally occurring, and non-naturally occurring, and which have similar binding properties as the reference nucleic acid. Examples of such analogs and/or modified residues include, without limitation, phosphorothioates, phosphoramidates, locked nucleic acids (LNAs), methyl phosphonates, chiral-methyl phosphonates, 2′-O-methyl ribonucleotides, and peptide-nucleic acids (PNAs).

The term “nucleic acid” includes any polynucleotide, wherein a polynucleotide may have any length of two or more nucleotides. A deoxy-ribooligonucleotide consists of a 5-carbon sugar called deoxyribose joined covalently to phosphate at 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally occurring bases, sugars and intersugar (backbone) linkages. The term “polynucleotide” can also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases. It should be understood that the term “polynucleotide” can also include polymers or oligomers comprising both deoxy and ribonucleotide combinations or variants thereof in combination with backbone modifications, such as those described herein.

The “nucleic acid” described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino 3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates).

The term “primer” is used to describe a single-stranded polynucleotide that is at least partially complementary to a target polynucleotide. A primer may be 10-50 nucleotides. A primer is generally configured for use in an amplification reaction, wherein the primer anneals to the target polynucleotide and is enzymatically extended. The end product of an amplification reaction may be a double-stranded target polynucleotide or a single-stranded target polynucleotide.

The term “amplification” is used in this disclosure to describe production of polynucleotides from a template polynucleotide. Amplification may be exponential, such as polymerase chain reaction (PCR) or it may be linear. Any suitable enzyme, reaction components, and thermal conditions may be used. An “amplification cycle” as used in this disclosure refers to extension of a single set of templates, e.g., for PCR, one amplification cycle includes one denaturation step, one annealing step, and one extension step. Annealing may occur at multiple temperatures. Amplification may comprise any suitable number of cycles, most typically between one and 40 cycles.

The term “immobilized” is used in this disclosure to describe that a molecule has been attached to a substrate. The immobilized molecule may be covalently attached to the substrate, or it may be otherwise strongly attached (e.g., it may be attached by a biotin-avidin linkage). An immobilized molecule may or may not dissociate from a substrate under given reaction conditions or in buffers of interest, for example, in physiological conditions.

The term “uniform” is used in this disclosure to describe a population of polynucleotides that are substantially identical to each other. As used herein, polynucleotides that are “substantially identical” to another polynucleotide is a polynucleotide sequence that has 70% or more sequence identity to the other polynucleotide sequence. As used herein, at least 90% of the polynucleotides in a “uniform” population of polynucleotides are substantially identical.

The term “annealing” is used in this disclosure to describe two complementary nucleotide sequences forming a double-stranded structure by nucleobase pairing.

The term “complementary” is used throughout this application to describe two related nucleic acid sequences that may form a double-stranded complex of a first 3′ to 5′ “top” strand and a second 5′ to 3′ “bottom” strand. Alternately, a double-stranded complex may include a first 5′ to 3′ “top” strand and a 3′ to 5′ “bottom” strand. A sequence may form base pairs with at least 70%, at least 80%, or at least 90% of the nucleotides of a complementary sequence. When a polynucleotide contains a complementary sequence, it should be understood that the complementary sequence may only be a portion of the polynucleotide.

The term “error” used in the context of nucleic acids throughout this disclosure refers to an inclusion or exclusion of a nucleotide component in a sequence that is not present in the intended design of the sequence. An “error” may refer to the site of a single nucleotide, or it may refer to the site of multiple nucleotides. Errors may include abasic sites, gaps, mismatches, deletions, undesired nucleotide modifications, incorporation of incorrect backbone components such as ribose or modified phosphate groups, or incorporation of extra nucleotides. “Gaps” may include sites at which one or more nucleotides are missing. Errors may be incorporated during chemical or enzymatic synthesis of a nucleic acid, or errors may occur after synthesis, e.g., by contaminating enzymes or chemicals.

Reference throughout this disclosure to “embodiments,” “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this disclosure are not necessarily referring to the same embodiment of the disclosure. Furthermore, the particular embodiments, including features, configurations, compositions, or characteristics may be combined in any suitable manner in one or more embodiments.

### BRIEF DESCRIPTION OF DRAWINGS

FIG. 1 is a general illustrative representation of annealing and ligation of multiple polynucleotides to a polynucleotide attached to a solid support.

FIG. 2A shows a general illustrative embodiment of treatment of a double-stranded polynucleotide including a mismatched base pair with an enzyme to cause a double-stranded break at the site of the mismatched base pair.

FIG. 2B shows a general illustrative embodiment of treatment of a double-stranded polynucleotide including a gap with an enzyme to cause a double-stranded break at the site of the gap.

FIG. 2C shows a general illustrative embodiment of treatment of a double-stranded polynucleotide without errors with an enzyme to cause a double-stranded break at the site of any errors. As no errors are present, no double-stranded break is produced.

FIG. 3 shows a general illustrative embodiment of a solid support with an immobilized single-stranded polynucleotide template and a solid support with an immobilized double-stranded polynucleotide template.

FIG. 4 shows a general illustrative representation of production of a single-stranded polynucleotide using an immobilized single-stranded polynucleotide template and a general illustrative method of production of a double-stranded polynucleotide using an immobilized single-stranded polynucleotide template.

FIG. 5 shows a general illustrative method of production of a double-stranded polynucleotide by annealing two single-stranded polynucleotides.

FIG. 6 shows a DNA gel of single-stranded and double-stranded polynucleotides.

FIG. 7 shows a Bioanalyzer™ gel image with DNA that detached from a bead solid state during different conditions in an assembly step.

FIG. 8 shows a DNA gel that demonstrates detachment of DNA from a glass solid state during assembly steps.

FIG. 9 shows a DNA gel that illustrates non-specific adsorption of DNA onto a silanized glass surface during the assembly steps.

FIG. 10 shows a DNA gel that illustrates non-specific adsorption of DNA on a bead surface during the assembly steps.

FIG. 11 shows a Bioanalyzer gel image with specific DNA assembly on streptavidin coated PCR tubes in the presence of a biotin-labeled anchor polynucleotide. Lanes 1-12 show: (1) Sample 1 purified, (2) Sample 2 purified, (3) Sample 3 purified, (4) Sample 4 purified, (5) Sample 5 1:10 dilution, (6) Sample 7 1:10 dilution, (7) Sample 5, (8) Sample 7, 9) Sample 1, (10) Sample 1 1:10 dilution, (11) Sample 3, and (12) Sample 3 1:10 dilution.

FIG. 12 shows a DNA gel with anchor-specific assembly of different genes assembled using streptavidin coated PCR tubes.

Schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION

Production of polynucleotides is an important tool for many different areas of biotechnology, including recombinant protein production, basic biochemical studies, use as therapeutics, and data storage.

The present disclosure describes methods of producing double-stranded and single-stranded polynucleotides. In embodiments, polynucleotides may be linear. In embodiments where a population of more than one polynucleotide is produced, the population of polynucleotides may be uniform. In embodiments, these methods include the production of error-corrected immobilized single-stranded or double-stranded polynucleotide templates. The present disclosure additionally describes an error-corrected immobilized single-stranded or double-stranded polynucleotide template.

In embodiments, the immobilized single-stranded polynucleotide template may include ribonucleic acid (RNA) nucleotides, deoxyribonucleic acid (DNA) nucleotides, or xenonucleic acid (XNA) nucleotides. XNA nucleotides may include any nucleotides that have a different sugar backbone than DNA or RNA. XNA may include 1,5-anhydrohexitol nucleic acid, cyclohexene nucleic acid, threose nucleic acid, glycol nucleic acid, locked nucleic acid, peptide nucleic acid, fluoro arabino nucleic acid, or other synthetic nucleotide analogues not described herein. The immobilized single-stranded polynucleotide template may additionally include modified nucleotides. Modified nucleotides may include 2′-O-methyl, 2′-O-methoxy-ethyl DNA, 2′-O-methyl RNA, fluoronucleotides, 2-aminopurine nucleotides, 5-bromo deoxyuridine, deoxy-2,6-diaminopurines, dideoxy-cytosine, hydroxymethyl deoxycytidine, inverted deoxynucleotides, iso-deoxynucleotides, 5-methylnucleotides, 5-nitroindole nucleotides, not limited to, deoxynucleotides, nucleotides with fluorophores, such as 6-carboxyfluorescein (FAM), cyanine-based dyes, ATTO, or ALEXA FLUOR dyes, nucleotides with added 3′ or 5′ phosphate groups, nucleotides with added amino groups, biotinylated nucleotides, nucleotides with a thiol group, alkyne modified nucleotides, adenylated nucleotides, nucleotides with azide groups, and nucleotides with other chemical linkers.

In embodiments, the immobilized polynucleotide template may include non-nucleoside modifications. Non-nucleoside modifications may refer to any chemical modifications which may be incorporated into a sugar-phosphate backbone but do not include a nucleobase. Examples of non-nucleoside modifications include, but are not limited to, aliphatic linkers and intentionally abasic sites. Any additional suitable chemical moieties described herein may be incorporated as non-nucleoside modifications.

In embodiments, the immobilized single-stranded polynucleotide template may include backbone modifications. Backbone modifications may include, but are not limited to incorporation of photocleavable linkers such as [4-(4,4′-Dimethoxytrityloxy) butyramidomethyl)-1-(2-nitropheny 1)-ethyl]-2-cyanoethyl-(N,N-diisopropy 1)-phosphoramidite and 3-(4,4′-Dim ethoxytrity 1)-1-(2-nitropheny 1)-propan-1-yl-[(2-cyanoethyl)-(N,N-diisopropyl)]-phosphoramidite, and/or incorporation of polyethylene glycol linkers of any suitable length.

In embodiments, the immobilized polynucleotide template may include one or more regions of purposefully randomized nucleotides. Randomized nucleotides may be desired if multiple variants of a region will be produced. For example, if a population of immobilized polynucleotide templates includes a sequence for a given protein, a sequence encoding a region of the protein to be investigated using saturating mutagenesis may include randomized nucleotides to result in a population of double-stranded immobilized polynucleotides with each possible codon combination in the region of the protein to be investigated. In other embodiments, none of the nucleotides of the immobilized polynucleotide template may be randomized.

The length of the immobilized polynucleotide templates is typically dependent upon the length of the annealed polynucleotides and the number of annealed polynucleotides used.

The polynucleotides and methods described herein are typically treated with an enzyme to remove any errors. As such, the polynucleotides described herein typically have fewer errors than polynucleotides prepared without treatment with such an enzyme. In some embodiments, a method of the present disclosure yields a population of polynucleotides including fewer errors than a population of polynucleotides produced using a comparable method not including treating with an enzyme to remove errors.

In some embodiments, a population of the polynucleotides described herein have an increased sequence homogeneity relative to a population of polynucleotides produced using a method not including treating with an enzyme to remove errors. As it is used herein, “sequence homogeneity” refers to the percentage of sequences within a population having an identical sequence across a given window.

### Production of an Immobilized Polynucleotide Template

In embodiments, a method for producing the immobilized polynucleotide template includes providing a solid support including an attached polynucleotide, wherein the attached polynucleotide is attached to the solid support; and annealing multiple polynucleotides to the attached polynucleotide to yield a double-stranded immobilized polynucleotide assembly comprising annealed polynucleotides. This double-stranded immobilized polynucleotide assembly includes a top strand with a discontinuous phosphate backbone and a bottom strand with a discontinuous phosphate backbone, wherein the top strand is attached to the solid support. In some embodiments, the method then includes treating the double-stranded immobilized polynucleotide assembly with a ligase to create a double-stranded immobilized polynucleotide. The multiple polynucleotides of the top strand are ligated to result in the top strand having a continuous phosphate backbone and the multiple polynucleotides of the bottom strand are ligated to result in the bottom strand having a continuous phosphate backbone. In some embodiments, the method does not include treating the double-stranded immobilized polynucleotide assembly with a ligase. In some of these embodiments, the top and bottom strands of the double-stranded immobilized polynucleotide assembly remain discontinuous. These discontinuous strands may be referred to as “splints” and may be desirable in some applications.

In some embodiments, the method includes treating the double-stranded immobilized polynucleotide with an enzyme to cause a double-stranded break at errors present between the two strands of the double-stranded immobilized polynucleotide to yield an error-corrected double-stranded immobilized polynucleotide.

In some embodiments, the error-corrected double-stranded immobilized polynucleotide template is used as an amplification template.

In some embodiments, the error-corrected double-stranded immobilized polynucleotide may be denatured and the bottom strand may be removed to yield an immobilized single-stranded polynucleotide template. In some embodiments, the immobilized single-stranded polynucleotide is used as an amplification template. Herein, the term “immobilized polynucleotide template” is used to refer to the immobilized single-stranded polynucleotide template, the double-stranded polynucleotide template, or both. One of skill in the art will understand that many properties that apply to one of the single-stranded or double-stranded templates should apply to the other.

The solid support may be glass, silicon oxide, metal, metal oxides, polymer, or plastic or any combination thereof. The solid support may have a shape designed to facilitate its use in creating the immobilized polynucleotide template. In embodiments, the solid support may be a pane of material, a slide, a flow cell, one or more beads, a single tube, a strip tube, a multi-well plate, a porous membrane, a chromatography membrane, a resin, a filter, a frit, or an insoluble gel. In embodiments where the solid support is one or more beads, the beads may be paramagnetic. The beads may be packed in a column compatible with chromatography. The solid support may be provided in a housing defining a reaction space. Production of the immobilized polynucleotide template can take place in the reaction space. After production of the immobilized polynucleotide template, further reaction such as amplification may take place in the reaction space.

The solid support comprising an attached polynucleotide may be provided by multiple sources. In embodiments, the solid support with an attached polynucleotide may be produced by obtaining an unmodified solid support and attaching one or more polynucleotides to the solid support.

In embodiments, the attached polynucleotide may be at least 10, at least 20, at least 40, at least 60, or at least 200 nucleotides in length. In embodiments, the attached polynucleotide may be at most 300, at most 500, or at most 1000 nucleotides in length. The attached polynucleotide may be attached to the solid support by 3′ end, or by 5′ end. In embodiments, the point of attachment to the solid support is within 30 nucleotides, within 20 nucleotides, within 10 nucleotides, within 5 nucleotides, within 3 nucleotides, or within one nucleotide of either end of the polynucleotide.

Methods for making a polynucleotide are known in the art and include, but are not limited to, enzymatic synthesis and chemical synthesis.

In embodiments, a polynucleotide may be modified to facilitate attachment to the solid support. The modification may be on 3′ end or 5′ end of the polynucleotide. Typically, the modification is on 5′ end. The modification may be added during chemical synthesis, or it may be added after the polynucleotide has been synthesized. In embodiments, the modification may be an amino group, a biotin group, a desthiobiotin group, a thiol, an alkyne group, a phosphate group, an H-phosphonate group, a glyceryl group, a dinitrophenol group, a phosphoramidite group, or an azide group. The modification may be further reacted to yield a moiety compatible with attachment. The attached polynucleotide may include chemical extensions (e.g., PEG or an alkane chain) of any suitable length. The chemical extension may be between a nucleotide of the polynucleotide and the modification.

The attached polynucleotide may be attached to the solid support at 3′ end, at 5′ end, or between 3′ and 5′ ends.

Methods for attaching a polynucleotide to a solid support are known in the art. In embodiments, the attached polynucleotide is attached to the solid support by a covalent attachment. Examples of reactions that may facilitate covalent attachment include, but are not limited to, thiol-maleimide, H-phosphonate, phosphoramidite, NETS-ester, isocyanate, isothiocyanate, benzoyl fluoride, diazonium, iodoacetamide, pyridoxal phosphate, Staudinger ligation, copper-catalyzed azide, strain promoted azide cycloaddition, or oxime ligation reaction. In embodiments, the attached polynucleotide is attached to the solid support by a non-covalent attachment. Examples of non-covalent attachment include, but are not limited to, a biotin-streptavidin interaction, a peptide affinity interaction (e.g., FLAG tag interaction), fluorous affinity-binding interaction, or a Ni-NTA-based interaction. In embodiments, the attached polynucleotide may be attached by adsorption. In embodiments, the attached polynucleotide may be synthesized directly on the solid support.

In some embodiments, the attached polynucleotide is reversibly attached to the solid support. In this way, if the attached polynucleotide becomes detached from the surface, it may reattach to surface. Typically, if the polynucleotide is detached from the surface, it may reattach to the surface with or without intervention. In other words, when the polynucleotide is attached to the surface via an affinity interaction, if the polynucleotide becomes detached from the surface, it can reattach when the proper conditions for attachment are restored. For example, in an embodiment wherein the polynucleotide is attached to the surface via a streptavidin-biotin interaction, if the streptavidin-biotin attachment is broken, it can reassemble when the biotin and streptavidin come into contact.

The present disclosure describes that a reversible, noncovalent attachment may improve retention of immobilized polynucleotides and allow for the recapture of polynucleotides with universal adapter or linker sequences. Without wishing to be bound by theory, it is believed that when a covalently bound polynucleotide is detached from a surface, it typically does not covalently reattach to the surface. Thus, if the surface including a population of covalently attached polynucleotides is washed, whatever portion of the polynucleotides has detached are permanently lost. In contrast, if a polynucleotide is attached to a surface via a reversible attachment, it may rebind to the surface, allowing for recapture of the molecule. A population of polynucleotides may migrate around a surface as they attach to and detach from a surface. However, methods of the present disclosure rely less upon stable localization of attached polynucleotides than on bulk retention of a population of polynucleotides.

In embodiments, at least one, at least 10, at least 100, at least 1000, at least 10,000, at least 100,000, at least 1,000,000 at least 10,000,000, at least 100,000,00 polynucleotides may be attached to the solid support. In embodiments, at most 50,000,000, at most 100,000,000, or at most 1,000,000,000 polynucleotides may be attached to the solid support. In embodiments where more than one polynucleotide is attached to the solid support, the sequence of the attached polynucleotides may be at least 70%, at least 80%, at least 90%, or at least 95% identical. Alternately, in embodiments each attached polynucleotide may be designed with a different sequence.

In embodiments, at least one femtomole, at least one picomole (pm), at least 10 pm, at least 100 pm, at least one nanomole (nm), at least 10 nm, at least 100 nm, at least one micromole at least 10 μm, or at least 100 μm of polynucleotide may be attached to the solid support. In embodiments, at most 10 millimoles (mm), at most 1 mm, at most 100 μm, or at most 10 μm of polynucleotide may be attached to the solid support.

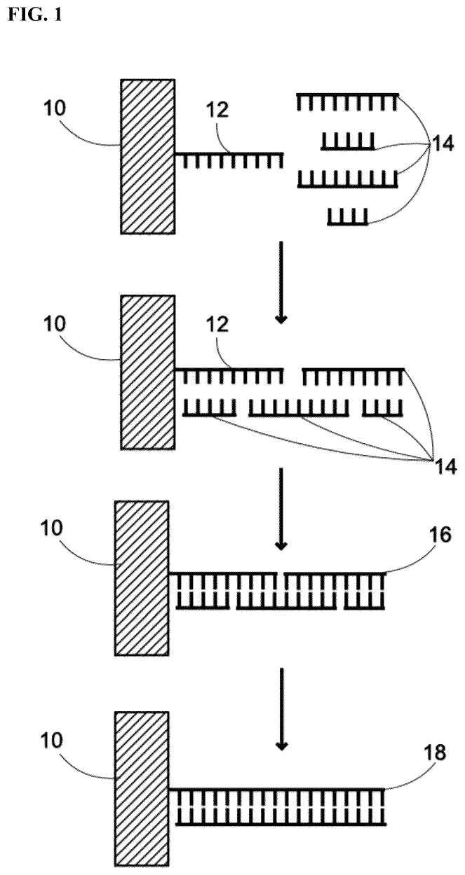

A method of the present disclosure includes annealing multiple polynucleotides to the attached polynucleotide to yield a double-stranded immobilized polynucleotide assembly having annealed polynucleotides. When multiple polynucleotides are annealed to the attached polynucleotide, it should be understood that not every annealed polynucleotide will form base pairs with the attached polynucleotide. The annealing may be visualized as an assembly of polynucleotides forming a top strand that forms base pairs with a bottom strand as shown in FIG. 1. A solid support 10 including an attached polynucleotide 12 is combined with multiple polynucleotides 14 to be annealed. The attached polynucleotide 12 and multiple polynucleotides 14 anneal in a brick-like assembly to form a double-stranded immobilized polynucleotide assembly 16. In embodiments, at least 3, at least 10, at least 20, or at least 50 polynucleotides may be annealed.

The conditions of annealing may be modified to promote proper assembly of the double-stranded immobilized polynucleotide assembly 16 and prevent formation of undesired hairpins or other secondary structure. In embodiments, the multiple polynucleotides 14 are provided in equimolar ratios. In embodiments, the multiple polynucleotides 14 are provided in non-equimolar ratios. In embodiments, the annealing may take place in a formulated buffer. The buffer may include water, magnesium chloride, Tris salts, phosphate, sodium chloride, sodium citrate, EDTA, or a combination thereof. The buffer may be or may be substantially similar to Tris buffered saline (TBS) or phosphate buffered saline (PBS). The buffer may have any suitable pH. The multiple oligonucleotides 14 may be designed to anneal at high temperatures to prevent formation of undesired secondary structure. During annealing, the temperature may gradually decrease over time. To begin the annealing process, the reaction space may be heated to a high temperature to remove any preexisting secondary structure. The reaction space may be heated to a temperature of at least 40° C., at least 50° C., at least 60° C., or at least 80° C. The reaction space may be heated to a temperature of at most 110° C. In embodiments, multiple annealing cycles may be completed to improve base pairing fidelity. In embodiments wherein multiple annealing cycles are completed, the polynucleotides used in each annealing cycle may be different. In embodiments wherein multiple annealing cycles are completed, the polynucleotides used in an annealing cycle may be different. In embodiments wherein multiple annealing cycles are completed, the polynucleotides used in an annealing cycle may be the same. After the multiple polynucleotides 14 have been annealed, the reaction space may be washed with buffer to remove any excess polynucleotides and other reaction components.

The double-stranded immobilized polynucleotide assembly 16 includes a top strand, wherein the top strand includes the attached polynucleotide and annealed polynucleotides, and a bottom strand, wherein the bottom strand includes multiple polynucleotides annealed to the top strand polynucleotides. The double-stranded immobilized polynucleotide assembly 16 includes a discontinuous phosphate backbone as shown in FIG. 1

The phosphate backbone may be ligated to yield a double-stranded immobilized polynucleotide 18 including a top strand with a continuous phosphate backbone and a bottom strand with a continuous phosphate backbone. In embodiments, the double-stranded immobilized polynucleotide assembly 16 may be treated with a ligase for at least 10 minutes, at least 30 minutes, or at least 60 minutes. In one or more embodiments, the double-stranded immobilized polynucleotide assembly 16 may be treated with a ligase for at most 180 minutes. In embodiments, the ligase is a Taq-based ligase. In embodiments, treating with a ligase includes treating with more than one ligase. The buffer in the reaction space may be exchanged with a buffer formulated for ligase activity prior to treating with a ligase. The buffer formulated for ligase activity may include divalent metal cations, ATP, reducing agents, or a combination thereof. In embodiments, treating with a ligase may include treating with a ligase in addition to one or more enzymes, such as a phosphatase or a kinase. The ligase and enzyme used may be modified to account for reaction parameters, such as the length of the double-stranded immobilized polynucleotide assembly 16, the reaction temperature, or the source of the annealed polynucleotides. Following ligation, the reaction space may be washed with buffer to remove ligase, enzyme, and additional reaction components. Additional aspects of the ligation reaction may be modified to accommodate the source of the annealed polynucleotides, the length of the double-stranded immobilized polynucleotide assembly, and the length of the immobilized polynucleotide template to be produced.

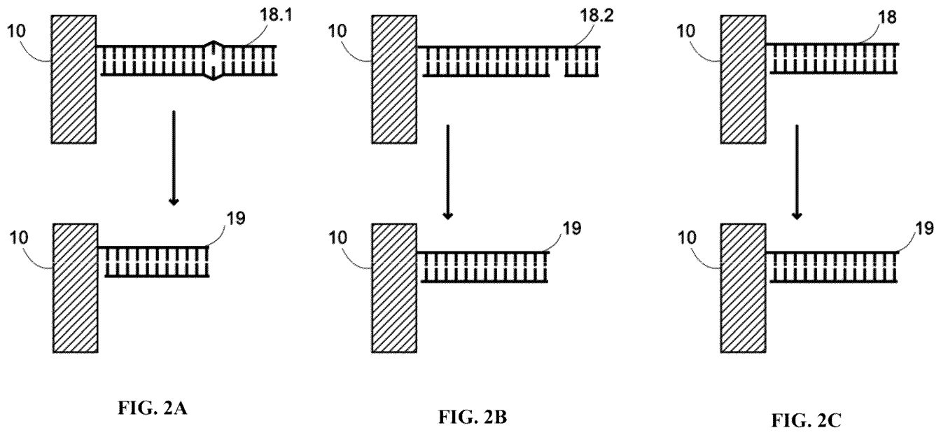

If the multiple polynucleotides have all been perfectly synthesized, every base in the theoretical double-stranded immobilized polynucleotide 18 would be paired to a corresponding base and the backbone would not contain any errors in the sugar or phosphate groups. However, it is possible that the experimental double-stranded immobilized polynucleotide 18 may contain one or more errors. An error may have been introduced during production of the multiple polynucleotides 14, or it may have been introduced after production (e.g., nucleotides may have been removed by a contaminating exonuclease or endonuclease). The error may include a mismatched base pair, an abasic site, a gap, an insertion, a deletion, a site with a nicked backbone, a chemical error in a nucleotide, a chemical error in a portion of the backbone, an error not described herein, or a combination thereof. At this stage, it is desired to remove a double-stranded immobilized polynucleotide 18 that contains one or more errors. In embodiments where more than one immobilized polynucleotide template is produced, removing an error in a double-stranded immobilized polynucleotide 18, or removing the error-containing double-stranded immobilized polynucleotide 18 (e.g., FIG. 2A-C, 18.1, 18.2) may improve the quality of the resulting population of immobilized polynucleotide templates. In embodiments, the quality of the population of immobilized polynucleotide templates may be measured as the percent identity between the sequence of the designed immobilized polynucleotide templates and the sequence of the experimentally produced immobilized polynucleotide templates. In embodiments where the population of immobilized polynucleotide templates are designed to have the same sequence, the quality of the population may be measured as the uniformity of the population of immobilized polynucleotide templates.

In embodiments, the double-stranded immobilized polynucleotide 18 may be treated with an enzyme to remove any errors. In embodiments, the enzyme may introduce a double-stranded cut at the site of the error. In embodiments, the double-stranded immobilized polynucleotide 18 may be treated with more than one enzyme to remove any errors.

In some embodiments, the enzyme is a nuclease. In some embodiments, the nuclease is an endonuclease. In some embodiments, the endonuclease is an apurinic endonuclease, an apyrimidinic endonuclease, or a restriction endonuclease.

In some embodiments, the enzyme has methylase activities. In some embodiments, the enzyme acts on double-stranded DNA. In some embodiments, the enzyme acts on single-stranded DNA. Typically, the enzyme is not a sequence-specific enzyme. However, the enzyme typically recognizes a particular feature of a nucleic acid, such as an error. In some embodiments, the enzyme recognizes nucleotide mismatches. In some embodiments, the enzyme recognizes apurinic sites. In some embodiments, the enzyme recognizes apyrimidinic sites. In some embodiments, the enzyme recognizes abasic sites. In some embodiments, the enzyme recognizes single-stranded nucleic acid. In some of these embodiments, the enzyme degrades single-stranded nucleic acid. In some embodiments, the enzyme recognizes modified nucleotides, such as methylated nucleotides.

When an enzyme “recognizes” a particular sequence or feature, the enzyme typically acts on or proximate to that feature. For example, an enzyme that recognizes a methylated nucleotide may excise that base or introduce a cut to the backbone on one or both sides of the methylated nucleotide. In some preferred embodiments, an enzyme introduces a double-stranded break at or near the site of a DNA mismatch.

In some embodiments, the enzyme may be or may include APE1, endonuclease II, endonuclease IV, endonuclease V, endonuclease VIII, Fpg, hAAG, hSMUG1, mismatch endonuclease I, T4 PDG, T7 endonuclease I, or a combination thereof.

FIG. 2 shows an illustration of several possible errors corrected by treatment with an enzyme. For example, in FIG. 2A, a double-stranded immobilized polynucleotide including a mismatched base pair 18.1 is cleaved at the site of the mismatch, resulting in an error-corrected double-stranded immobilized polynucleotide 19. In another example FIG. 2B, a double-stranded immobilized polynucleotide including a gap 18.2 is cleaved at the site of the gap, resulting in an error-corrected double-stranded immobilized polynucleotide 19.

In some embodiments, errors may not be detected at this point. In some other embodiments, errors may be detected at this point. There may be more than one error present. There may alternately be no errors present. In embodiments, it is assumed that at least one error is present, and the double-stranded immobilized polynucleotide 18 may be treated with one or more enzymes to remove any potential errors. Experimentally, if no errors are present, treatment with the one or more enzymes may not impact the double-stranded immobilized polynucleotide 18 as shown in FIG. 2C. Independent of the number and type of errors present in the double-stranded immobilized polynucleotide 18, the product of treating the double-stranded immobilized polynucleotide 18 with an enzyme may be referred to as the error-corrected double-stranded immobilized polynucleotide 19. After treating with an enzyme, the reaction space may be washed with buffer to remove reaction components.

The error-corrected double-stranded immobilized polynucleotide 19 may include a top strand and a bottom strand, wherein each strand has a continuous phosphate backbone, and the top strand is attached to the solid support. In embodiments, the error-corrected double-stranded immobilized polynucleotide 19 is denatured to separate the top and bottom strands. In embodiments, denaturing may include increasing temperature, treating with a chemical, treating with strong base, or a combination thereof. In some of these embodiments, the strong base may be sodium hydroxide. In some of these embodiments, the temperature may be increased using a gradient. In embodiments, denaturing may include adding dimethyl sulfoxide to the reaction space. In embodiments, denaturing may include enzymatic denaturing or degrading. Any suitable denaturing method, including those described herein, combinations of those described herein, or methods not described herein may be used.

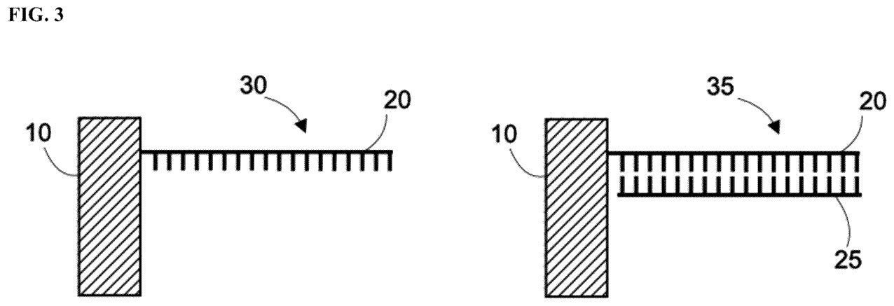

After the top strand and the bottom strand have been denatured, the reaction space may be washed with buffer to remove the bottom strand. The top strand, which is attached to the solid support and remains in the reaction space, may then be referred to as the immobilized single-stranded polynucleotide template, represented by 20 in FIG. 3. In this context, the term “template” is used to denote the idea that the immobilized single-stranded polynucleotide is suitable for enzymatic amplification with a primer.

In embodiments, the immobilized single-stranded polynucleotide template may be detached from the solid support. If the desired product is a single-stranded polynucleotide, production and subsequent harvest of the template may yield the desired product. The immobilized single-stranded polynucleotide template may be enzymatically detached (e.g., using a restriction enzyme). In embodiments where the immobilized single-stranded polynucleotide template is attached to the solid support by an affinity interaction, a competing affinity molecule may be added to detach the single-stranded polynucleotide template. The immobilized single-stranded polynucleotide template may be removed by chemical or heat treatment to break the attachment to the solid support. After detachment, the reaction space may be washed to remove the detached single-stranded polynucleotide template.

The method of producing a polynucleotide template as described herein is compatible with production of templates of different lengths. The length of the template produced typically depends on the length of the annealed polynucleotides used for production of the double-stranded immobilized polynucleotide assembly. Embodiments describing two ranges of lengths of single-stranded immobilized polynucleotide templates that may be produced are described here.

### Production of Templates Up to 4,000 Nucleotides in Length

In some embodiments, the produced single-stranded polynucleotide template may be up to 4,000 nucleotides in length. In embodiments, the annealed polynucleotides may be chemically o enzymatically synthesized. In embodiments, the annealed polynucleotides may be enzymatically synthesized. The synthesized polynucleotides may have a length of at least 10 nucleotides, at least 20 nucleotides, at least 40 nucleotides, at least 60 nucleotides, or at least 200 nucleotides. In embodiments, the synthesized polynucleotides may have a length of at most 300 nucleotides, at most 300 nucleotides, at most 500 nucleotides, or at most 1000 nucleotides.

When the annealed polynucleotides are chemically synthesized, the length of the double-stranded immobilized polynucleotide assembly is typically limited by the limitations of chemical synthesis. In embodiments where the multiple polynucleotides are chemically synthesized, the double-stranded immobilized polynucleotide assembly may be at least 40 nucleotides, at least 60 nucleotides, at least 80 nucleotides, at least 100 nucleotides, at least 500 nucleotides, at least 1000 nucleotides, or at least 2,000 nucleotides in length. In embodiments, the double-stranded immobilized polynucleotide assembly may be at most 2,000 nucleotides or at most 4,000 nucleotides in length.

### Production of Templates Up to 20,000 Nucleotides

In some embodiments, the produced single-stranded polynucleotide template may be up to 20,000 nucleotides in length. The assembled polynucleotides may have been produced by amplifying a first round of immobilized single-stranded polynucleotide templates. In these embodiments, the polynucleotides may be at least 100 nucleotides, at least 200 nucleotides, or at least 300 nucleotides in length. In embodiments, the polynucleotides may be at most 1,000 nucleotides, at most 600 nucleotides, or at most 500 nucleotides in length. When the assembled polynucleotides have been produced by amplifying a first round of immobilized single-stranded polynucleotide templates, the length of the double-stranded immobilized polynucleotide assembly may be much greater than when the polynucleotides are chemically synthesized. In embodiments, the length of the double-stranded immobilized polynucleotide template may be at least 2,000, at least 5,000, at least 10,000, or at least 20,000 nucleotides in length. In some of these embodiments, the double-stranded immobilized polynucleotide assembly may be at most 10,000 nucleotides, at most 15,000 nucleotides, or at most 20,000 nucleotides in length.

In embodiments, the polynucleotides may include a combination of chemically synthesized polynucleotides and polynucleotides produced by amplifying a first round of immobilized single-stranded polynucleotide templates.

### Production of a Single-Stranded Polynucleotide

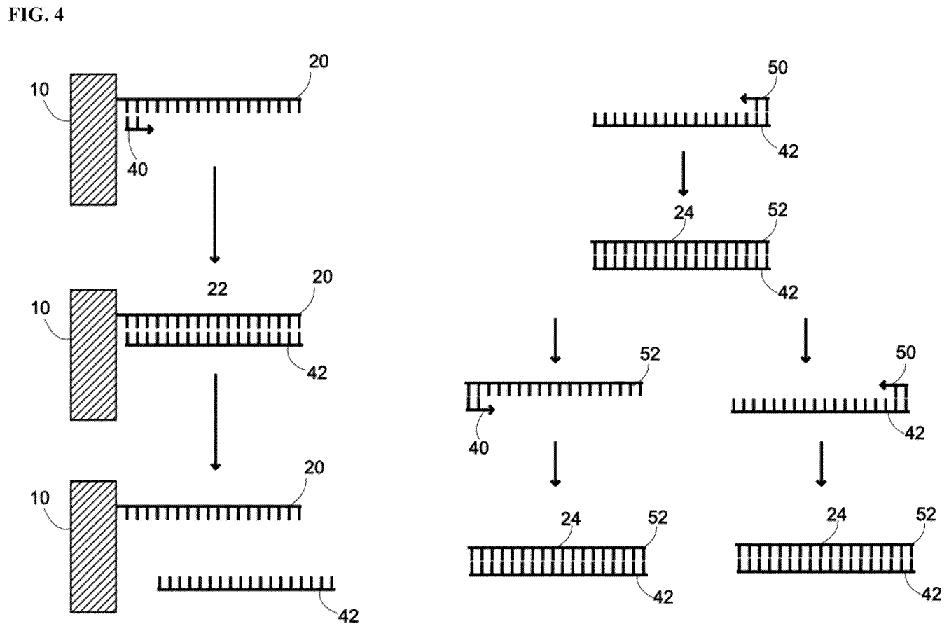

In embodiments, the immobilized single-stranded polynucleotide template 20 may be used to produce a single-stranded polynucleotide as shown in FIG. 4. In embodiments, a method for producing a single-stranded polynucleotide using the immobilized single-stranded polynucleotide template 20 includes amplifying the immobilized single-stranded polynucleotide template 20 enzymatically with a primer 40 to result in an immobilized double-stranded polynucleotide 22. The top strand of the immobilized double-stranded polynucleotide 22 is the immobilized single-stranded polynucleotide template 20 and the bottom strand 42 of the immobilized double-stranded polynucleotide 22 includes the primer 40. At least a portion of the immobilized polynucleotide 22 may be double-stranded. The method then includes denaturing the immobilized double-stranded polynucleotide 22 to result in a bottom strand polynucleotide 42 and the immobilized single-stranded polynucleotide template 20 and removing the bottom strand polynucleotide 42 from the immobilized single-stranded polynucleotide template 20 to yield a single-stranded polynucleotide 42.

Typically, the length of the single-stranded polynucleotide is determined by the length of the immobilized single-stranded polynucleotide template and the location of the primer 40 used in its production. In embodiments, the immobilized single-stranded polynucleotide template used to produce the single-stranded polynucleotide was prepared from chemically synthesized polynucleotides. In some of these embodiments, the single-stranded polynucleotide has a length of at least 100 nucleotides, at least 200 nucleotides, or at least 400 nucleotides. In embodiments, the single-stranded polynucleotide has a length of at most 2,000 nucleotides, at most 1,000 nucleotides, or at most 800 nucleotides. In some of these embodiments, the single-stranded polynucleotide has a length of at least 1,000 nucleotides, at least 2,000 nucleotides, or at least 4,000 nucleotides. In some of these embodiments, the single-stranded polynucleotide has a length of at most 20,000 nucleotides, at most 10,000 nucleotides, or at most 8,000 nucleotides.

Multiple methods of amplification are compatible with amplification of the immobilized single-stranded polynucleotide template. In embodiments, amplifying the immobilized single-stranded polynucleotide template includes a single amplification cycle. In embodiments, amplifying the immobilized single-stranded polynucleotide template includes more than one amplification cycle. Depending on the desired composition of the single-stranded polynucleotide, different enzymes and reaction conditions may be used to amplify the immobilized single-stranded polynucleotide template. Amplification of the immobilized single-stranded polynucleotide template may be exponential, or it may not be exponential.

The primer used during amplification may have any suitable length and composition. The primer may be at least two, at least five, at least 10, or at least 20 nucleotides in length. The primer may be at most 100, at most 80, at most 60, or at most 40 nucleotides in length. The calculated annealing temperature of primer may be about 45° C. to about 80° C.

In embodiments, the immobilized single-stranded polynucleotide template may be amplified using polymerase chain reaction (PCR). In embodiments, the immobilized single-stranded polynucleotide template is amplified using isothermal amplification, such as loop-mediated isothermal amplification (LAMP) or recombinase polymerase amplification (RPA). In embodiments, the immobilized single-stranded polynucleotide template is amplified using a DNA polymerase. In some of these embodiments, the DNA polymerase is a Taq-based polymerase or a family A polymerase. The DNA polymerase may have been engineered, for example, to incorporate non-natural nucleotides. In embodiments, the immobilized single-stranded polynucleotide template is amplified by an RNA polymerase. In some of these embodiments, the RNA polymerase is T7 polymerase. In embodiments, the immobilized single-stranded polynucleotide template is amplified by a reverse transcriptase. In some of these embodiments, the reverse transcriptase may be Moloney murine leukemia virus reverse transcriptase. In embodiments, the immobilized single-stranded polynucleotide template may be amplified by an RNA-dependent RNA polymerase. In some of these embodiments, the RNA-dependent RNA polymerase may be T7 RNA-dependent RNA polymerase. The amplification reaction may include any suitable components such as dNTPs, metal ions, DTT, spermidine, ATP, cofactors, or a combination thereof. The amplification reaction may include any suitable buffer components such as Tris-HCl, salts, water or a combination thereof.

In embodiments, a designed mutation may be introduced into the single-stranded polynucleotide. A mutation may be introduced by amplifying the immobilized single-stranded polynucleotide template with a primer that includes at least one nucleotide not complementary to the immobilized single-stranded polynucleotide template.

Following amplification, the enzymes and other components used during amplification may be inactivated using heat, chemicals, or a combination thereof. Reaction components may be removed from the reaction space using any suitable method.

In embodiments, the immobilized single-stranded polynucleotide template may be regenerated for future use after the top and bottom strands are dissociated and the single-stranded polynucleotide has been removed. The immobilized single-stranded polynucleotide template may be regenerated by washing with buffer, by treating with heat, or by treating enzymatically. After regeneration, the immobilized single-stranded polynucleotide template maybe reused and regenerated again.

In embodiments, the method for producing a single-stranded polynucleotide further includes purifying the single-stranded polynucleotide after it has been removed from the immobilized single-stranded polynucleotide template. In embodiments, purifying the single-stranded polynucleotide may include chromatographic purification, spin column-based purification (e.g., using a kit such as a Qiagen DNA cleanup kit), ethanol precipitation, gel purification, magnetic bead purification, or a combination thereof.

In embodiments, rather than denaturing the immobilized double-stranded polynucleotide 22 and removing the bottom strand polynucleotide, the immobilized double-stranded polynucleotide 22 may be detached from the solid support and collected. In embodiments where the desired product is a double-stranded polynucleotide, detaching the immobilized double-stranded polynucleotide 22 may yield the desired product. The immobilized double-stranded polynucleotide 22 may be detached from the solid support chemically or enzymatically as described herein.

### Production of an Amplified Double-Stranded Polynucleotide

In another aspect, this disclosure describes a method of amplifying an immobilized polynucleotide template to yield an amplified double-stranded polynucleotide.

In some embodiments, the double-stranded immobilized polynucleotide 25 is amplified to produce an amplified double-stranded polynucleotide. In some embodiments, the complementary strand of the immobilized double-stranded polynucleotide may be used as a template for amplification.

Alternatively, the complementary strand 25 can be removed prior to amplification. The complementary strand 25 can be removed to yield a single-stranded immobilized polynucleotide. In some embodiments, the single-stranded immobilized polynucleotide 20 is amplified to produce an amplified double-stranded polynucleotide.

In some embodiments, a method of making a double-stranded polynucleotide includes amplifying the single-stranded polynucleotide 20 with a first primer 40 and a second primer 50 multiple times to produce an amplified template 24. The resulting amplified template 24 can be enzymatically treated, for instance with a 3′-5′ exonuclease, to break down incompletely amplified polynucleotides and excess primers. Following this treatment, the amplified template from reaction components to produce an amplified double-stranded polynucleotide (FIG. 4).

The length of the amplified double-stranded polynucleotide is typically dependent on the length of the single-stranded polynucleotide used in its production. In embodiments, the amplified double-stranded polynucleotide may have a length of at least 100 nucleotides, at least 200 nucleotides, at least 400 nucleotides, at least 1,000 nucleotides, at least 2,000 nucleotides, or at least 8,000 nucleotides. In embodiments, the amplified double-stranded polynucleotide has a length of at most 20,000 nucleotides, at most 10,000 nucleotides, at most 8,000 nucleotides, at most 2,000 nucleotides, at most 1,000 nucleotides, or at most 800 nucleotides.

The amplified double-stranded polynucleotide may include DNA, RNA, and/or XNA bases. In embodiments, the amplified double-stranded polynucleotide may include nucleotide modifications as described herein.

In embodiments, amplifying the single-stranded polynucleotide may include any of the amplification methods described herein. In some embodiments, the first primer, the second primer, or both may introduce one or more intended mutations into the amplified double-stranded polynucleotide.

In embodiments, treating the amplified template with 3′-5′ exonuclease comprises treating with E. coli Exonuclease I, III or V, RecJf exonuclease, Exonuclease T, or a combination thereof.

In embodiments, the method for producing an amplified double-stranded polynucleotide further includes purifying the amplified double-stranded polynucleotide. Purifying the amplified double-stranded polynucleotide may include any of the purification methods described herein.

### Production of an Assembled Double-Stranded Polynucleotide

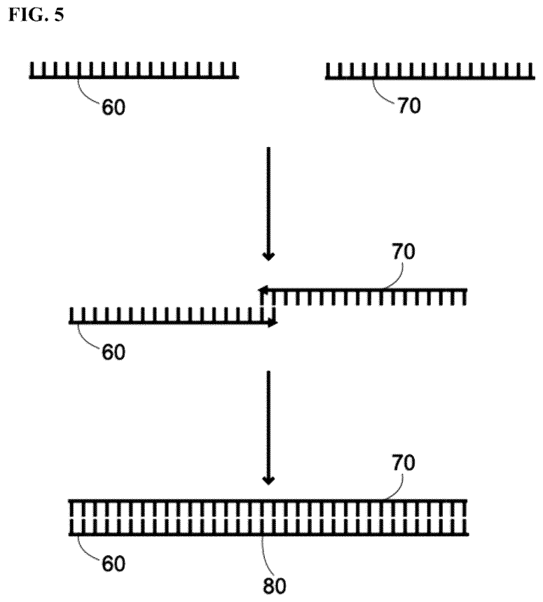

In another aspect, the present disclosure describes a method of producing a double-stranded polynucleotide by partially annealing two different single-stranded polynucleotides 42 and extending each strand. In embodiments, a method for producing an assembled double-stranded polynucleotide includes providing a first single-stranded polynucleotide 60 including a complementary region of nucleotides on 3′ end which are complementary to the 3′ end of a second single-stranded polynucleotide 70. The number of complementary nucleotides can be, for instance, from 10 to 20 nucleotides. The method includes annealing the first single-stranded polynucleotide 60 and second single-stranded polynucleotide 70 to produce a partially annealed assembly, extending the partially annealed assembly to produce a double-stranded polynucleotide 80, and amplifying the double-stranded polynucleotide to produce an assembled double-stranded polynucleotide (FIG. 5). Shorter fragments of DNA may be created during the production of each single-stranded polynucleotide may be complementary to 5′ end of either polynucleotide but not long enough to overlap at 3′ end of the complementary polynucleotide. These shorter fragments may be used as primers to create a double-stranded full-length polynucleotide while simultaneously removing otherwise unwanted incomplete assembly products.

In embodiments, the complementary region of nucleotides on 3′ end of the first single-stranded polynucleotide 60 may be any suitable number of nucleotides. In embodiments, the complementary region may include at least two, at least four, at least six, at least eight, at least 10, at least 15, at least 20, at least 25, or at least 30 complementary nucleotides. In embodiments, the complementary region may include at most 200, at most 100, at most 80, at most 70, at most 50, at most 40, or at most 30 complementary nucleotides.

The length of the assembled double-stranded polynucleotide is typically dependent on the length of the single-stranded polynucleotides used in its production. In embodiments, the assembled double-stranded polynucleotide may have a length of 200 to 4,000 nucleotides, 400 to 2,000 nucleotides, or 800 to 1,600 nucleotides. In embodiments, the amplified double-stranded polynucleotide may have a length of at least 200 nucleotides, at least 400 nucleotides, at least 600 nucleotides, at least 800 nucleotides, at least 2,000 nucleotides, at least 4,000 nucleotides, at least 8,000 nucleotides, or at least 10,000 nucleotides. In embodiments, the amplified double-stranded polynucleotide may have a length of at most 8,000 nucleotides, at most 10,000 nucleotides, at most 16,000 nucleotides, at most 20,000 nucleotides, or at most 40,000 nucleotides.

The assembled double-stranded polynucleotide may include DNA, RNA, and/or XNA bases. In embodiments, the amplified double-stranded polynucleotide may include nucleotide modifications as described herein.

In embodiments, extending each strand of the partially annealed assembly includes amplification with any of the polymerases described herein. Extending each strand of the partially annealed assembly may be isothermal, or it may include multiple temperatures. The assembled double-stranded polynucleotide may be purified using any of the methods described herein.

### Immobilized Single-Stranded Polynucleotide Template as a Product.

During or following the practice of the methods described herein, a number of compounds and compositions may result. For example, a compound or composition including a solid support including an immobilized single-stranded polynucleotide template may result. A representation of the solid support including an immobilized single-stranded polynucleotide template 30 is shown in FIG. 3. The solid support including an immobilized single-stranded polynucleotide template 30 includes a solid support 10 and an immobilized single-stranded polynucleotide template 20.

The solid support may include any of the features or compositions described herein. In embodiments, the solid support may be provided in a suitable form for shipping or storage. In embodiments, the immobilized single-stranded polynucleotide template may be at least 40, at least 60, at least 100, at least 200, at least 400, at least 1,000, at least 2,000, at least 5,000, at least 8,000, at least 10,000, at least 12,000, at least 15,000, or at least 20,000 nucleotides in length. In embodiments, the immobilized single-stranded polynucleotide template may be at most 50,000, at most 30,000, at most 20,000, or at most 10,000 nucleotides in length.

In embodiments, the solid support may include at least one, at least ten, at least 50, at least 100, at least 1,000, at least 10,000, at least 100,000, or at least 1,000,000 immobilized single-stranded polynucleotide templates. In embodiments, the solid support may include at most 1,000,000,000, at most 50,000,000, or at most 25,000,000 immobilized single-stranded polynucleotide templates. In some of these embodiments, the immobilized single-stranded polynucleotide templates may have sequences that are at least 75%, at least 85%, or at least 95% identical. In embodiments, the solid support may include immobilized single-stranded polynucleotide templates with at least 10, at least 100, or at least 1,000 different sequences. In embodiments, the solid support may include immobilized single-stranded polynucleotide templates with at most 1,000,000, at most 500,000, or at most 100,000 different sequences. In embodiments, each of the immobilized single-stranded polynucleotide templates of a solid support has a different sequence.

The solid support and immobilized single-stranded polynucleotide may include any suitable features described herein, including those describing size, characteristics, composition, and function. The immobilized single-stranded polynucleotide template may include any nucleotides described herein, including DNA, RNA, XNA, and modified nucleotides. The immobilized single-stranded polynucleotide template may include regions of randomized nucleotides as described herein.

### Formulation as a Kit

The present disclosure also provides a kit for amplification of a solid support including an immobilized single-stranded polynucleotide template. In embodiments, the kit includes the solid support including an immobilized single-stranded polynucleotide template, one or more enzymes for amplification, one or more enzymes for error correction, primers, suitable buffers, or a combination thereof. In embodiments, the kit includes materials for purification of an amplified polynucleotide product. In embodiments, the kit is compatible for use with a liquid handling robot or another automated lab machine.

Optionally, other reagents such as buffers or a pharmaceutically acceptable carrier (either prepared or present in its constituent components, where one or more of the components may be premixed or all of the components may be separate), and the like, are also included. In one embodiment, the solid support may be present with a buffer, or may be present in separate containers. Instructions for use of the packaged components are also typically included.

As used herein, the phrase “packaging material” refers to one or more physical structures used to house the contents of the kit. The packaging material is constructed by known methods, preferably to provide a sterile, contaminant-free environment. The packaging material has a label, which indicates that the contents can be used for amplification of the solid support. In addition, the packaging material contains instructions indicating how the materials within the kit are used.

As used herein, the term “package” refers to a solid matrix or material such as glass, plastic, paper, foil, and the like, capable of holding within fixed limits a solid support including an immobilized single-stranded polynucleotide. Thus, for example, a package can include a glass or plastic vial used to contain appropriate quantities of primers, buffers, or enzymes. “Instructions for use” typically include a tangible expression describing the reagent concentration or at least one method parameter.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1 is a method for producing an immobilized error-corrected double-stranded polynucleotide template, the method comprising: providing a solid support comprising an attached polynucleotide, wherein the attached polynucleotide is attached to the solid support; annealing multiple polynucleotides to the attached polynucleotide to yield a double-stranded immobilized polynucleotide assembly comprising annealed polynucleotides, wherein the double-stranded immobilized polynucleotide assembly comprises a top strand with a discontinuous phosphate backbone and a bottom strand with a discontinuous phosphate backbone, wherein the top strand is attached to the solid support; treating the double-stranded immobilized polynucleotide assembly with a ligase to create a double-stranded immobilized polynucleotide, wherein the multiple polynucleotides of the top strand are ligated to result in the top strand comprising a continuous phosphate backbone and the multiple polynucleotides of the bottom strand are ligated to result in the bottom strand comprising a continuous phosphate backbone; treating the double-stranded immobilized polynucleotide with an enzyme to remove an error in the double-stranded immobilized polynucleotide, yielding an immobilized error-corrected double-stranded polynucleotide template.

Aspect 1.5 is the method of aspect 1, further comprising denaturing the double-stranded immobilized polynucleotide; and removing the bottom strand to yield an immobilized single-stranded polynucleotide template.

Aspect 2 is the method of aspect 1, wherein providing a solid support comprising an attached polynucleotide comprises attaching polynucleotides to a solid support.

Aspect 3 is the method of aspect 1, wherein providing a solid support comprising an 30 attached polynucleotide comprises obtaining the solid support comprising an attached polynucleotide.

Aspect 4 is the method of any one of aspects 1 to 3, wherein the attached polynucleotide is attached to the support by a covalent attachment.

Aspect 5 is the method of aspect 4, wherein the covalent attachment is facilitated by thiol-maleimide, H-phosphonate, phosphoramidite, NETS-ester, isocyanate, isothiocyanate, benzoyl fluoride, diazonium, iodoacetamide, reductive amination, Diels-Alder, Mitsunobu reaction, carbodiimides and other peptide bond-forming reagents, pyridoxal phosphate, Staudinger ligation, Suzuki coupling, copper-catalyzed azide, strain promoted azide cycloaddition, or oxime ligation reaction.

Aspect 6 is the method of any one of aspects 1 to 3, wherein the attached polynucleotide is attached to the support by a non-covalent attachment.

Aspect 7 is the method of aspect 6, wherein the non-covalent attachment comprises a biotin-avidin attachment, a fluorous attachment, or an antibody attachment.

Aspect 8 is the method of any one of aspects 1 to 7, wherein the solid support comprises a pane, a slide, a flow cell, one or more beads, a multi-well plate, a tube, a strip tube, a porous membrane, or an insoluble gel.

Aspect 9 is the method of any one of aspects 1 to 8, wherein the solid support comprises glass, metal, silicon oxide, metal oxides, polymer, or plastic.

Aspect 10 is the method of any one of aspects 1 to 9, wherein the attached polynucleotide is attached by 3′ end.

Aspect 11 is the method of any one of aspects 1 to 9, wherein the attached polynucleotide is attached by 5′ end.

Aspect 12 is the method of any one of aspects 1 to 11, wherein the attached polynucleotide is attached by a non-terminal nucleotide.

Aspect 13 is the method of any one of aspects 1 to 12, wherein the attached polynucleotide and/or one or more of the multiple polynucleotides are chemically synthesized.

Aspect 14 is the method of any one of aspects 1 to 13, wherein the attached polynucleotide and/or one or more of the multiple polynucleotides are enzymatically synthesized.

Aspect 15 is the method of any one of aspects 1 to 14, wherein the annealed polynucleotides are prepared by amplification of a first immobilized single-stranded polynucleotide template.

Aspect 16 is the method of any one of aspects 1 to 15, wherein annealing multiple polynucleotides to the attached polynucleotide comprises annealing at least 3 polynucleotides.

Aspect 17 is the method of aspect 16, wherein annealing multiple polynucleotides to the attached polynucleotide comprises annealing at least 10 polynucleotides.

Aspect 18 is the method of aspect 17, wherein annealing multiple polynucleotides to the attached polynucleotide comprises annealing at least 50 polynucleotides.

Aspect 19 is the method of any one of aspects 1 to 18, wherein the annealed polynucleotides are between 20 and 2000 nucleotides.

Aspect 20 is the method of aspect 19, wherein the annealed polynucleotides are between 40 and 200 nucleotides.

Aspect 21 is the method of aspect 20, wherein the annealed polynucleotides are between 60 and 100 nucleotides.

Aspect 22 is the method of any one of aspects 1 to 21, wherein the annealed polynucleotides are provided in equimolar ratios.

Aspect 23 is the method of any one of aspects 1 to 21, wherein the annealed polynucleotides are provided in non-equimolar ratios.

Aspect 24 is the method of any one of aspects 1 to 23, wherein annealing comprises annealing by decreasing a reaction temperature.

Aspect 25 is the method of any one of aspects 1 to 24, wherein treating the double-stranded immobilized polynucleotide assembly with a ligase comprises treating with a Taq-based ligase. Aspect 26 is the method of any one of aspects 1 to 25, wherein treating the double-stranded immobilized polynucleotide assembly with a ligase comprises treating with more than one ligase.

Aspect 27 is the method of any one of aspects 1 to 26, wherein the double-stranded polynucleotide assembly is discontinuous.

Aspect 28 is the method of any one of aspects 1 to 27, wherein treating the double-stranded immobilized polynucleotide with an enzyme comprises treating with APE1, endonuclease II, endonuclease IV, endonuclease V, endonuclease VIII, Fpg, hAAG, hSMUG1, mismatch endonuclease I, T4 PDG, T7 endonuclease I, or a combination thereof.

Aspect 29 is the method of any one of aspects 1 to 28, wherein treating the double-stranded immobilized polynucleotide with an enzyme comprises treating with more than one enzyme. Aspect 30 is the method of any one of aspects 1 to 29, wherein the error is a mismatched base pair, an abasic site, a site with a nicked backbone, a gap, a chemical error in a nucleotide, a chemical error in a portion of the backbone, or a combination thereof.

Aspect 31 is the method of any one of aspects 1 to 30, wherein treating the double-stranded immobilized polynucleotide with an enzyme results in a double-stranded break at a site of the error.

Aspect 32 is the method of aspects 1 to 31, wherein treating the double-stranded immobilized polynucleotide with an enzyme results in correction of the error.

Aspect 33 is the method of any one of aspects 1 to 32, wherein the attached polynucleotide and/or one or more of the multiple polynucleotides comprises RNA nucleotides, DNA nucleotides, XNA nucleotides, LNA nucleotides, or a combination thereof.

Aspect 34 is the method of any one of aspects 1 to 33, wherein the attached polynucleotide and/or one or more of the multiple polynucleotides comprises a backbone modification.

Aspect 35 is the method of any one of aspects 1 to 34, wherein the attached polynucleotide and/or one or more of the multiple polynucleotides comprises modified nucleotides.

Aspect 36 is the method of any one of aspects 1 to 35, wherein the attached polynucleotide and/or the one or more of multiple polynucleotides comprises non-nucleoside modifications.

Aspect 37 is the method of any one of aspects 1 to 36, wherein the immobilized double-stranded or single-stranded polynucleotide template has a length of at least 100 nucleotides, at least 200 nucleotides, at least 500 nucleotides, or at least 1,000 nucleotides.

Aspect 38 is the method of any one of aspects 1 to 37, wherein immobilized double-stranded or single-stranded polynucleotide template has a length of at most 2,000 nucleotides, at most 5,000 nucleotides, or at most 20,000 nucleotides.

Aspect 39 is the method of any one of aspects 1 to 38, wherein the attached polynucleotide and/or one or more of the annealed polynucleotides comprises a region of purposefully randomized bases.

Aspect 40 is a method for producing a single-stranded polynucleotide, the method comprising: amplifying the immobilized double-stranded or single-stranded polynucleotide template of any one of aspects 1 to 39 enzymatically with a primer to result in an immobilized double-stranded polynucleotide, wherein the top strand of the immobilized double-stranded polynucleotide comprises the immobilized single-stranded polynucleotide template and the bottom strand of the immobilized double-stranded polynucleotide comprises the primer, wherein at least a portion of the immobilized double-stranded polynucleotide is double-stranded; denaturing the immobilized double-stranded polynucleotide to result in a bottom strand polynucleotide and the immobilized single-stranded polynucleotide template; and collecting the bottom strand polynucleotide to yield a single-stranded polynucleotide.

Aspect 41 is the method of aspect 40, wherein the single-stranded polynucleotide has a length of at least 40 nucleotides, at least 100 nucleotides, at least 200 nucleotides, at least 500 nucleotides, or at least 1,000 nucleotides.

Aspect 42 is the method of any one of aspects 40 or 41, wherein the single-stranded polynucleotide has a length of at most 2,000 nucleotides, at most 5,000 nucleotides, or at most 20,000 nucleotides.

Aspect 43 is a method for producing double-stranded polynucleotides, the method comprising: amplifying the immobilized double-stranded or single-stranded polynucleotide template of any one of aspects 1 to 39 enzymatically with two primers, to result in an amplified template, wherein amplifying comprises multiple rounds of amplification, treating the amplified template with 3′-5′ exonuclease; and separating the amplified template from reaction components to yield double-stranded polynucleotides.

Aspect 44 is the method of aspect 43, wherein the double-stranded polynucleotide has a length of at least 100 nucleotides, at least 200 nucleotides, at least 500 nucleotides, or at least 1,000 nucleotides.

Aspect 45 is the method of any one of aspects 43 or 44, wherein the double-stranded polynucleotide has a length of at most 2,000 nucleotides, at most 5,000 nucleotides, or at most 10,000 nucleotides.

Aspect 46 is the method of any one of aspects 43 to 45, wherein amplifying the immobilized polynucleotide template comprises a single amplification cycle.

Aspect 47 is the method of any one of aspects 43 to 46, wherein amplifying the immobilized polynucleotide template comprises more than one amplification cycle.

Aspect 48 is the method of any one of aspects 43 to 47, wherein amplifying the immobilized polynucleotide template comprises single-direction amplification.

Aspect 49 is a method for producing an assembled double-stranded polynucleotide, the method comprising: obtaining two of the single-stranded polynucleotides of any one of aspects 1 to 39, wherein a first single-stranded polynucleotide comprises at least 20 nucleotides at the 3′ end that are complementary to the 3′ end of a second single-stranded polynucleotide; annealing the first single-stranded polynucleotide and the second single-stranded polynucleotide to produce a partially annealed assembly; extending the partially annealed assembly to produce a double-stranded polynucleotide; and amplifying the double-stranded polynucleotide to produce an assembled double-stranded polynucleotide.

Aspect 50 is the method of aspect 49, wherein the assembled double-stranded polynucleotide has a length of at least 500 nucleotides, at least 1,000 nucleotides, at least 2,000 nucleotides, or at least 10,000 nucleotides.

Aspect 51 is the method of any one of aspects 49 or 50, wherein the assembled double-stranded polynucleotide has a length of at most 20,000 nucleotides, at most 30,000 nucleotides, or at most 50,000 nucleotides.

Aspect 52 is the method of any one of aspects 43 to 51, wherein amplifying comprises amplification by a DNA polymerase.

Aspect 53 is the method of aspect 52, wherein the DNA polymerase comprises a Taq polymerase.

Aspect 54 is the method of any one of aspects 43 to 53, amplifying comprises amplification by an RNA polymerase.

Aspect 55 is the method of aspect 54, wherein the RNA polymerase comprises a T7 polymerase.

Aspect 56 is the method of any one of aspects 40 to 55, wherein amplifying comprises amplification by a reverse transcriptase.

Aspect 57 is the method of aspect 56, wherein the reverse transcriptase comprises Moloney murine leukemia virus reverse transcriptase.

Aspect 58 is the method of any one of aspects 40 to 57, wherein amplifying comprises amplification by an RNA-dependent RNA polymerase.

Aspect 59 is the method of aspect 58, wherein the RNA-dependent RNA polymerase comprises a T7 RNA polymerase.

Aspect 60 is the method of any one of aspects 40 to 59, wherein the primer comprises at least one nucleotide that is not complementary to the immobilized polynucleotide template.

Aspect 61 is the method of any one of aspects 40 to 60, wherein the method further comprises regenerating the immobilized polynucleotide template to be used repeatedly.

Aspect 62 is the method of any one of aspects 40 to 61, wherein only a portion of the immobilized polynucleotide template is amplified.

Aspect 63 is the method of any one of aspects 1 to 39 or 52 to 62, wherein the method further comprises purifying the immobilized polynucleotide template.

Aspect 64 is the method of any one of aspects 40 to 43 or 52 to 62, wherein the method further comprises purifying the polynucleotide.

Aspect 65 is the method of any one of aspects 44 to 49 or 52 to 62, wherein the method further comprises purifying the double-stranded polynucleotides.

Aspect 66 is the method of any one of aspects 50 to 62, wherein the method further comprises purifying the assembled double-stranded polynucleotide.

Aspect 67 is the method of any one of aspects 63 to 66, wherein purifying comprises gel purification, column purification, precipitation, chromatography, or a combination thereof.

Aspect 68 is a solid support comprising one or more of the immobilized double-stranded or single-stranded polynucleotide template of any one of aspects 1 to 39.

Aspect 69 is the solid support of aspect 68 wherein the immobilized double-stranded or single-stranded polynucleotide template comprises at least 40 nucleotides.

Aspect 70 is the solid support of aspect 68 wherein the immobilized double-stranded or single-stranded polynucleotide template comprises at least 200 nucleotides.

Aspect 71 is the solid support of aspect 68 wherein the immobilized double-stranded or single-stranded polynucleotide template comprises at least 1,000 nucleotides.

Aspect 72 is the solid support of aspect 68 wherein the immobilized double-stranded or single-stranded polynucleotide template comprises at least 2,000 nucleotides.

Aspect 73 is the solid support of aspect 68 wherein the immobilized double-stranded or single-stranded polynucleotide template comprises at least 10,000 nucleotides.

Aspect 74 is the solid support of aspect 68 wherein the immobilized double-stranded or single-stranded polynucleotide template comprises at least 20,000 nucleotides.

Aspect 75 is the solid support of any one of aspects 68-74, wherein the solid support comprises a flow cell.

Aspect 76 is the solid support of any one of aspects 68-75, wherein the solid support comprises a bead.

Aspect 77 is the solid support of any one of aspects 68-76, wherein the solid support comprises a chip.

Aspect 78 is the solid support of any one of aspects 68-77, wherein the solid support comprises a membrane.

Aspect 79 is the solid support of any one of aspects 68-78, wherein the solid support comprises a microfluidic channel.

Aspect 80 is the solid support of any one of aspects 68-79, wherein the sequences of the one or more of the double-stranded or single-stranded polynucleotide templates are at least 75%, at least 80% or at least 90% identical.

Aspect 81 is the solid support of any one of aspects 68-79, wherein each of the one or more of the double-stranded or single-stranded polynucleotide templates comprises a different sequence.

Aspect 82 is the solid support of any one of aspects 68-79 and 81, wherein the one or more of the double-stranded or single-stranded polynucleotide template comprise at least 5, at least 10, at least 25, or at least 50 different sequences.

Aspect 83 is a kit comprising the solid support of any of aspects 68 to 82.

Aspect 84 is the kit of aspect 83, wherein the kit additionally comprises instructions for use, buffers, nucleotides, and consumables.

Aspect 85 is the kit of any one of aspects 83 or 84, wherein the kit is compatible for use with an automated device.

Aspect 86 is the method of any one of aspects 40 to 67, wherein amplifying comprises incorporating modified nucleotides.

### EXAMPLES

A summary of the reagents used in each experiment and the source of each reagent is described in Table 1.

TABLE 1
Reagents used in examples.

| Reagent | Full Name | Source |
|---|---|---|
| Streptavidin beads | Dynabeads ™ MyOne ™ | Thermo Fisher ™ |
| | Streptavidin Cl | Cat # 65001 |
| BcMag ™ beads | BcMag ™ beads Carboxyl- | Bioclone ™, Cat# FB-101 |
| | Terminated Magnetic Beads | |
| Glass Beads | Solid-glass beads, borosilicate, | Sigma-Aldrich ™ Cat # |
| | diam. 1 mm | Z273619-1EA |
| Streptavidin PCR Tubes | Streptavidin Coated PCR Tubes | Biomat ™ Code PCR0STF- |
| | | SA5/100 |
| Caps for PCR strip tubes | Caps for PCR strip tubes | Biomat ™ Code PCR 8 |
| HiFi ™ Ligase | HiFi ™ Taq DNA ligase | NEB ™ |
| | | Cat # M0647S |
| 10x ligase buffer | 10x Taq DNA ligase buffer | NEB ™ |
| | | Cat # M0647S |
| Sspl-HF | Sspl-HF Restriction enzyme | NEB ™ |
| | | Cat # R3132S |
| CutSmart ™ Buffer | CutSmart ™ Buffer | NEB ™ |
| | | Cat # R3132S |
| T7 Endonuclease | T7 Endonuclease I | NEB ™ Cat# M0302L |
| 20x SSC | SSC Buffer 20x Concentrate | Sigma-Aldrich ™ Cat #S6639- |
| | | 1L |
| SDS | SDS Solution, Molecular | Promega ™ V6551 |
| | Biology Grade (10% w/v) | |
| NaCl | Sodium Chloride, ACS, ≥99.0% | Sigma-Aldrich ™ Cat# S9888- |
| | | 500G |
| BSA | BSA-Molecular Biology Grade - | NEB ™ Cat# B9000S |
| | 12 mg | |
| T4 PNK Reaction Buffer | T4 Polynucleotide Kinase | NEB ™ Cat# B0201S |
| | Reaction Buffer | |
| T4 PNK Enzyme | T4 Polynucleotide Kinase | NEB ™ Cat# M0201S |
| T4 DNA Ligase | T4 DNA Ligase | NEB ™ Cat # M0202S |
| T4 DNA Ligase Reaction | T4 DNA Ligase Reaction | NEB ™ Cat # B0202S |
| Buffer | Buffer | |
| NaOH | Sodium Hydroxide | Sigma-Aldrich ™ |
| | | Cat # 1310-73-2 |
| 10 mM Tris Buffer, | Trizma Base | Sigma-Aldrich ™ |
| pH 7.5 | | Cat # T1503-250G |
| dNTPs | dNTP mix, 10 mM each | Promega ™ |
| | | Cat #U1511 |
| Taq polymerase | Taq polymerase, 5 U/μL | NEB ™ |
| | | Cat #M0273S |
| PCR Buffer | Standard 10x Taq Buffer | NEB ™ |
| | | Cat #M0273S |
| MgCl2 | Magnesium chloride | Sigma-Aldrich ™ |
| | hexahydrate | Cat # M2670-500G |
| Q5 polymerase | Q5 High-Fidelity DNA | NEB ™ |
| | Polymerase, 2 U/μL | Cat # M0491 S |
| Q5 PCR Buffer | Q5 5x Reaction Buffer | NEB ™ Cat # M0491S |
| Small RNA Bioanalyzer | Agilent ™ Small RNA | Agilent ™ 5067-15-18 |
| chip | Bioanalyzer kit | |
| Ss10 DNA ladder | Single stranded DNA ladder for | Simplex Sciences ™ |
| | small RNA Bioanalyzer ™ | |
| DNA Bioanalyzer Chip | Agilent ™ DNA 1000 Kit | Agilent ™ 5067-1504 |
| Agarose | Agarose, Certified Molecular | BioRad ™ Cat #1613101 |
| | Biology | |
| GelGreen ™ | GelGreen ™ Nucleic Acid Gel | Biotium ™ Cat #41005 |
| | Stain, 10,000x in water | |
| Low molecular weight | Low Molecular Weight DNA | NEB ™ Cat# N3233S |
| ladder | ladder | |
| 1 kb Plus DNA ladder | 1 kb Plus DNA ladder | NEB ™ Cat #N3200S |
| AMPure ™ beads | AMPure ™ XP Reagent | Beckman Coulter ™ Cat |
| | | #A63880 |

### Example 1

In example 1, two populations (“A” and “B”) of 350-400 nucleotide (nt) double-stranded immobilized polynucleotides were created using a method consistent with the processes described herein, including the methods depicted in FIG. 1 and FIG. 2. Each population was amplified, and the double-stranded product was removed from the reaction space.

To produce population A, a 46 nucleotide (nt) polynucleotide with a 3′ biotin modification was attached to a streptavidin-coated paramagnetic particle. The attached polynucleotide included 10 deoxythymidine residues, a six nt restriction enzyme recognition site, and the first 30 nt of the sequence to be assembled. Twelve polynucleotides ranging from 30-60 nt were designed so that each polynucleotide included at least one 30 nt region complementary to another polynucleotide, similarly to FIG. 1. Each polynucleotide was chemically synthesized. The multiple polynucleotides were combined in a 2:1 molar ratio of each free polynucleotide to the attached polynucleotide. The polynucleotides were prepared in a buffer including in 5 mM magnesium chloride. To anneal the polynucleotides, the temperature of the reaction was increased to 85° C. and subsequently decreased by 2° C. each minute until the temperature reached 53° C. After annealing, polynucleotides not annealed to the attached oligo were removed, and the solid support was washed with water and CutSmart™ buffer. HiFi™ DNA Taq ligate and HiFi™ Taq DNA Ligase Buffer were added to the annealed polynucleotides. The mixture was incubated at 55° C. for one hour to ligate the polynucleotides. Following ligation, the solid state was washed with water to remove reaction components and yield population A of double-stranded immobilized polynucleotides.

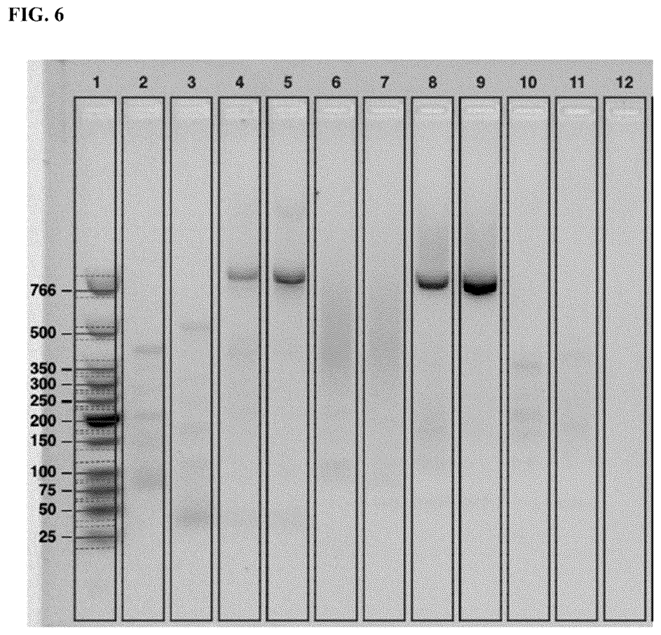

To produce population B, the process described above was repeated using a 44 nt attached polynucleotide and 13 polynucleotides having different sequences. The double-stranded immobilized polynucleotides from reactions A and B were cleaved from the solid state using Ssp1-HF, which was heat-inactivated following cleaving. The products of these reactions are shown in lanes 2 (population A) and 3 (population B) of FIG. 6. Bands correspond to population A (having a maximum length of 360 nt), population B (having a maximum length of 417 nt), and multiple shorter partially-assembled products for each of populations A and B.

### Example 2

In example 2, a double-stranded assembled polynucleotide was prepared as shown in FIG. 5 using the double-stranded immobilized polynucleotide populations A and B as described in Example 1.

Population A and population B of double-stranded immobilized polynucleotides are listed in Table 2 and were prepared as described in Example 1.

TABLE 2
Oligos used for assembly.

| | | SEQ |
|---|---|---|
| | | ID |
| Oligo Name | Oligo Sequence | NO |
| Population A Oligos | | |
| GFP Part 1A | /5Phos/ACC AGT GAA TAA TTC TTC ACC TTT AGA | 1 |
| | CAT AAT ATT TTT TTT TTT T/3Bio/ | |
| GFP A2 | /5Phos/GGA GAC AGA AAA TTT GTG ACC ATT AAC | 2 |
| | ATC ACC ATC TAA TTC AAC CAA AAT TGG GAC AAC | |
| GFP A3 | /5Phos/AGT AGT ACA AAT AAA TTT TAA GGT CAA | 3 |
| | TTT ACC GTA AGT AGC ATC ACC TTC ACC TTC ACC | |
| GFP A4 | /5Phos/ACA TTG AAC ACC ATA ACC GAA AGT AGT | 4 |
| | GAC TAA GGT TGG CCA TGG AAC TGG CAA TTT ACC | |
| GFP A5 | /5Phos/TTC TGG CAT GGC AGA CTT GAA AAA GTC | 5 |
| | ATG TTG TTT CAT ATG ATC TGG GTA TCT AGC AAA | |
| GFP A6 | /5Phos/AGC TCT GGT CTT GTA GTT ACC GTC ATC TTT | 6 |
| | GAA AAA AAT AGT TCT TTC TTG AAC ATA ACC | |
| GFP A7a | AAC TAA GGT ATC ACC TTC AAA CTT GAC TTC | 7 |
| GFP C1 | AAT ATT ATG TCT AAA GGT GAA GAA TTA TTC ACT GGT | 8 |
| | GTT GTC CCA ATT TTG GTT GAA TTA GAT GGT | |
| GFP C2 | /5Phos/GAT GTT AAT GGT CAC AAA TTT TCT GTC TCC GGT | 9 |
| | GAA GGT GAA GGT GAT GCT ACT TAC GGT | |
| GFP C3 | /5Phos/AAA TTG ACC TTA AAA TTT ATT TGT ACT ACT GGT | 10 |
| | AAA TTG CCA GTT CCA TGG CCA ACC TTA | |
| GFP C4 | /5Phos/GTC ACT ACT TTC GGT TAT GGT GTT CAA TGT TTT | 11 |
| | GCT AGA TAC CCA GAT CAT ATG AAA CAA | |
| GFP C5 | /5Phos/CAT GAC TTT TTC AAG TCT GCC ATG CCA GAA | 12 |
| | GGT TAT GTT CAA GAA AGA ACT ATT TTT TTC | |
| GFP C6 | /5Phos/AAA GAT GAC GGT AAC TAC AAG ACC AGA GCT | 13 |
| | GAA GTC AAG TTT GAA GGT GAT ACC TTA GTT | |
| F Primer Pt 1 | ATG TCT AAA GGT GAA GAA TTA TTC A | 14 |
| Population B Oligos | | |
| GFP Part 2A | /5Phos/CAT GGT ATG GAT GAA TTG TAC AAA TAA AAT | 15 |
| | ATT TTT TTT TTT TT/3Bio/ | |
| GFP A6 | /5Phos/AGC TCT GGT CTT GTA GTT ACC GTC ATC TTT GAA | 6 |
| | AAA AAT AGT TCT TTC TTG AAC ATA ACC | |
| GFP A7 | /5Phos/AAA ATC AAT ACC TTT TAA TTC GAT TCT ATT | 16 |
| | AAC TAA GGT ATC ACC TTC AAA CTT GAC TTC | |
| GFP A8 | /5Phos/AAC ATT GTG AGA GTT ATA GTT GTA TTC CAA | 17 |
| | TTT GTG ACC TAA AAT GTT ACC ATC TTC TTT | |
| GFP A9 | /5Phos/GTT GTG TCT AAT TTT GAA GTT AAC TTT GAT | 18 |
| | ACC ATT CTT TTG TTT GTC AGC CAT GAT GTA | |
| GFP A10 | /5Phos/ATC ACC AAT TGG AGT ATT TTG TTG ATA ATG GTC | 19 |
| | AGC TAA TTG AAC AGA ACC ATC TTC AAT | |
| GFP A11 | /5Phos/ATC TTT GGA TAA GGC AGA TTG AGT GGA TAA | 20 |
| | GTA ATG GTT GTC TGG TAA CAA GAC TGG ACC | |
| GFP A12 | TTA TTT GTA CAA TTC ATC CAT ACC ATG GGT AAT ACC | 21 |
| | AGC AGC AGT AAC AAA TTC TAA CAA GAC CAT GTG | |
| | GTC TCT CTT TTC GTT TGG | |
| GFP C6 | /5Phos/AAA GAT GAC GGT AAC TAC AAG ACC AGA GCT | 13 |
| | GAA GTC AAG TTT GAA GGT GAT ACC TTA GTT | |
| GFP C7 | /5Phos/AAT AGA ATC GAA TTA AAA GGT ATT GAT TTT | 22 |
| | AAA GAA GAT GGT AAC ATT TTA GGT CAC AAA | |
| GFP C8 | /5Phos/TTG GAA TAC AAC TAT AAC TCT CAC AAT GTT | 23 |
| | TAC ATC ATG GCT GAC AAA CAA AAG AAT GGT | |
| GFP C9 | /5Phos/ATC AAA GTT AAC TTC AAA ATT AGA CAC AAC | 24 |
| | ATT GAA GAT GGT TCT GTT CAA TTA GCT GAC | |
| GFP C10 | /5Phos/CAT TAT CAA CAA AAT ACT CCA ATT GGT GAT | 25 |
| | GGT CCA GTC TTG TTA CCA GAC AAC CAT TAC | |
| GFP C11 | /5Phos/TTA TCC ACT CAA TCT GCC TTA TCC AAA GAT CCA | 26 |
| | AAC GAA AAG AGA GAC CAC ATG GTC TTG | |
| GFP C12 | /5Phos/TTA GAA TTT GTT ACT GCT GCT GGT ATT ACC | 27 |
| F Primer Pt 2 | TTA TTT GTA CAA TTC ATC CAT ACC ATG | 28 |

The two strands of each double-stranded immobilized polynucleotide were denatured using NaOH and the non-immobilized strand was removed. The solid support was washed three times with neutralizing buffer including 10 mM Tris-HCl, pH 7.5 buffer, leaving the immobilized single-stranded polynucleotide template. Each population was amplified in a single-direction PCR cycle to produce a single-stranded polynucleotide A and a single-stranded polynucleotide B. The amplification reaction contained a final concentration of 1×PCR buffer, 2.5 units (U) of Taq polymerase, 5 mM additional MgCl2, 100 μM dNTPs and 400 nm F primer. Reactions were amplified according to Table 3. A portion of the products of these reactions was run on an agarose gel and stained with GelGreen™. These products can be observed as single-stranded smears in lanes 6 (population A) and 7 (population B) of FIG. 6.

TABLE 3
PCR Conditions.

| | Temperature | Time |
|---|---|---|
| 25 cycles | 95° C. | 15 seconds |
| | 55° C. | 30 seconds |
| | 68° C. | 60 seconds |

Equal parts of single-stranded polynucleotide pools A and B were combined with an equal part of fresh PCR master mix containing Q5™ PCR buffer, dNTPs, and Q5™ Taq polymerase in liquid state. Two replicates of this mixture were prepared, referred to herein as reactions C and D. Reactions C and D were prepared to test two modified polymerase protocols to extend the two strands.

Equal parts of each cut double-stranded polynucleotide pools A and B from Example 1 were also combined with an equal part of fresh PCR master mix containing Q5™ PCR buffer, dNTPs, and Q5™ Taq polymerase in liquid state. Two replicates of this mixture were prepared, referred to herein as reactions E and F.

The reactions in tubes C and E underwent 10 rounds of thermal cycling with each cycle consisting of 10 seconds at 98° C. and 1 minute at 72° C. The reactions in tubes D and F underwent 15 rounds of thermal cycling with each cycle consisting of 10 seconds at 98° C. and 1 minute at 72° C. The product of tube E is in lane 4 and the product of tube F is in lane 5 of FIG. 6. The product of tube C formed from reactions A and B is in lane 8 of FIG. 6 and the product of tube D formed from reactions A and B is in lane 9 of FIG. 6. Lanes 10-12 of the gel in FIG. 6, represent negative controls in which only reaction A (lane 10), only reaction B (lane 11), or only water (lane12) were added to the new reaction master mix. All of the negative controls failed to yield the desired product.

Each of reactions C, D, E, and F yielded a final product of the desired length, indicating that a product had been formed by annealing two single-stranded polynucleotides and extending to complete each strand. Shorter single-stranded oligos in the polynucleotide pools were also able to act as primers and were extended to form additional full-length product in the reaction with single-stranded inputs.

### Example 3

In this Example, the stability of the biotin-streptavidin linkage during standard PCR conditions was characterized. The polynucleotide was bound to a bead, PCR cycling was performed, and samples were taken from the solution to measure the presence of any polynucleotide that was no longer bound to the beads.

A 46 nucleotide (nt) attached polynucleotide was labeled on 3′ end with a biotin molecule (GFP Part 1A from Table 2) and was attached to the streptavidin-coated paramagnetic particle solid support Cl beads through biotin-streptavidin linkage. The beads were washed with 1×BW buffer (5 mm Tris-HCl, pH 7.5, 0.5 mM EDTA, and 1 M NaCl) and water and then resuspended in PCR buffers with various final added concentrations of MgCl2 to test the stability of the attached polynucleotide through different PCR conditions. Table 4 shows the final concentration of added MgCl2, PCR buffer type, and number of PCR cycles tested for each sample.

TABLE 4
Reaction conditions for polynucleotide stability test

| Sample | | Final added | Number of |
|---|---|---|---|
| number | PCR Buffer (1x) | MgCl2 (mM) | PCR cycles |
| 1 | Standard Taq | 0 | 30 |
| 2 | Standard Taq | 5 | 30 |
| 3 | Standard Taq | 10 | 30 |
| 4 | Standard Taq | 0 | 20 |
| 5 | Standard Taq | 5 | 20 |
| 6 | Standard Taq | 10 | 20 |
| 7 | Q5 | 5 | 30 |
| 8 | Q5 | 0 | 20 |
| 9 | Q5 | 5 | 20 |
| 10 | Water (no buffer) | 0 | 30 |

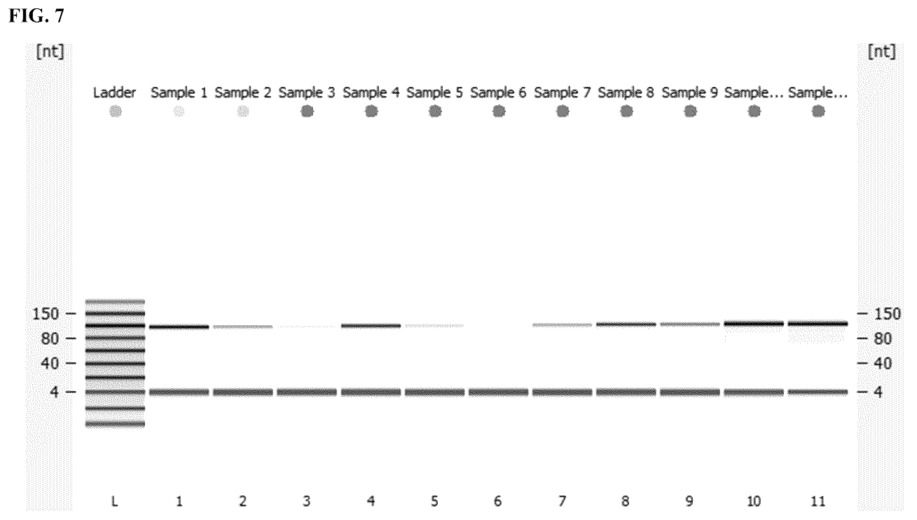

Following the thermocycling steps, the supernatant of each reaction was run on an Agilent Small RNA kit chip on a Bioanalyzer using a single stranded DNA ladder from Simplex Sciences™. The intensity of the bands on the gel image in FIG. 7 reflects the relative amount of polynucleotide that was lost during the thermal cycling process in each buffer composition. The samples are loaded in the order presented in the table above with an aliquot of the input polynucleotide (GFP Part 1A, Table 2) loaded as a positive control for Sample 11.

This data indicates that significant quantities of the polynucleotide did not remain attached to the Cl beads during PCR reaction conditions. Adding additional MgCl2 stabilized the biotin-streptavidin interaction and reduced but did not eliminate the loss of the attached polynucleotide.

### Example 4

In this Example, a functionalized ((N-(3-Triethoxysilylpropyl)-4-hydroxybutyramide)) glass bead solid state with a polynucleotide directly synthesized on the surface using standard 3′-phosphoramidite chemistry was evaluated for stability.

The sequence of the synthesized surface polynucleotide (“GFP Anchor Sequence”) is shown in Table 5 below. Table 5 also includes the sequences of the additional chemically prepared polynucleotides used for assembly. The polynucleotides used for assembly in this example were designed so that the anchored strand included 60-nt sequences and the complementary strand included 40-nt “splint” sequences, in which each polynucleotide overlapped the corresponding anchored strand polynucleotide segments by 20 nt.

TABLE 5
List of polynucleotides used in Example 4

| | | SEQ |
|---|---|---|
| Polynucleotide | | ID |
| Name | Polynucleotide Sequence | NO |
| GFP Anchor | ACC AGT GAA TAA TTC TTC ACC TTT AGA CAT | 29 |
| Sequence | GGT CGT GTT CAA TAT TCA TGT CTC GT | |
| GFP A2 | /5Phos/GGA GAC AGA AAA TTT GTG ACC ATT AAC | 2 |
| | ATC ACC ATV TAA. TIC AAC CAA AAT TGG GAC AAC | |
| GFP A3 | /5Phos/AGT AGT ACA AAT AAA TTT TAA GGT CAA TTT | 3 |
| | ACC GTA AGT AGC ATC ACC TTC ACC TTC ACC | |
| GFP A4 | /5Phos/ACA TTG AAC ACC ATA ACC GAA AGT AGT | 4 |
| | GAC TAA GGT TGG CCA TGG AAC TGG CAA TTT ACC | |
| GFP A5 | /5Phos/TTC TGG CAT GGC AGA CTT GAA AAA GTC ATG | 5 |
| | TTG TTT CAT ATG ATC TGG GTA TCT AGC AAA | |
| GFP C1s stop | TGA AGA ATT ATT CAC TGG TGT TGT CCC AAT | 30 |
| | TTT GGT TGA/3ddC/ | |
| GFP C2s stop | GTC ACA AAT TTT CTG TCT CCG GTG AAG GTG | 31 |
| | AAG GTG ATG/3ddC/ | |
| GFP C3s | TAA AAT TTA TTT GTA CTA CTG GTA AAT TGC | 32 |
| | CAG TTC CAT G | |
| GFP C4s | TCG GTT ATG GTG TTC AAT GTT TTG CTA GAT | 33 |
| | ACC CAG ATC A | |
| F primer | ACG AGA CAT GAA TAT TGA ACA | 34 |
| R primer | GAC TTG AAA AAG TCA TGT TG | 35 |

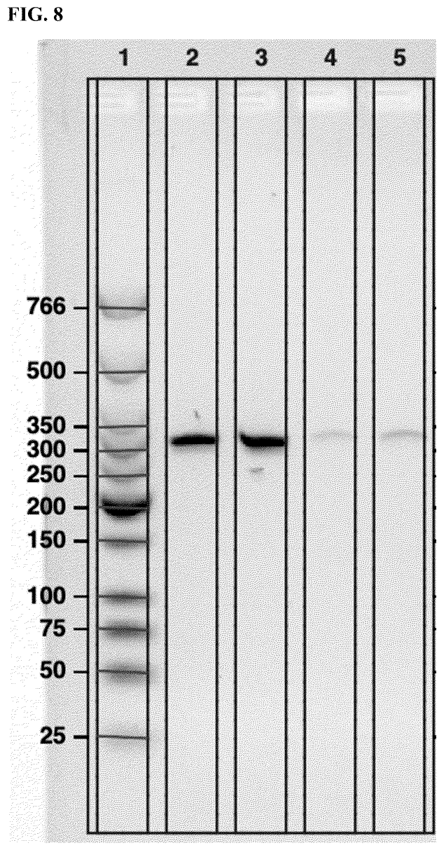

To prepare the synthesized polynucleotide on the glass surface for ligation, a phosphorylation reaction was performed using T4 polynucleotide kinase to add a phosphate to the 5′ end of the GFP anchor sequence. Following the completion of the phosphorylation reaction, the glass solid state was washed with water, and 100 picomoles of each polynucleotide for assembly and 2×SSC buffer (0.3 M NaCl, 0.03 M sodium citrate, pH 7.0.) were added to the reaction space. Annealing was achieved by increasing the temperature of the reaction to 80° C. and decreasing by 2° C. each minute until the temperature reached 46° C. Several washes were completed with 2× or 0.2×SSC with or without 0.1% SDS to remove polynucleotides that were not annealed to the anchored polynucleotide on the glass bead. Ligation was achieved by adding either T4 ligase and the corresponding T4 ligase buffer and incubating for 1 hour at 25° C. or by adding HiFi Taq DNA ligase and the corresponding HiFi Taq DNA ligase buffer and incubating for 1 hour at 48° C. The supernatant surrounding the glass beads was removed following the ligation reaction and was assayed with PCR to detect the polynucleotides that did not stay attached to the glass during ligation. 25 cycles of PCR were performed using the primers in Table 5, Q5 polymerase, Q5 buffer, and dNTPs. The agarose gel stained with GelGreen™ in FIG. 8 shows PCR products amplified from two replicate reactions post HiFi™ ligation (Lanes 2 and 3) and post T4 ligation (Lanes 4 and 5) with the low molecular weight ladder shown in Lane 1 for reference.

These data show that covalently anchored polynucleotides sometimes detached from the glass solid state during a ligation reaction and that the detachment was more likely under a higher temperature. This finding indicated that anchored polynucleotides were likely not compatible with a workflow that required a stably attached template.

### Example 5

In this Example, the potential for non-specific adsorption of polynucleotides in solution was evaluated for multiple types of beads. Beads evaluated included glass beads, silanized glass beads and silanized glass beads with a polynucleotide directly synthesized on the surface using standard 3′-phosphoramidite chemistry.

The sequence of the synthesized surface polynucleotide (“Anchor sequence”) is shown in Table 6 below. Table 6 also includes the sequences of the additional chemically prepared polynucleotides used for assembly on the solid state. The polynucleotides used for assembly in this example were designed so that the anchored strand included polynucleotides in 60-nt units and the complementary strand included polynucleotides in 60-nt units, in which each polynucleotide overlapped the corresponding anchored strand polynucleotide by 30-nt segments.

TABLE 6
List of polynucleotides used in Example 5

| | | SEQ ID |
|---|---|---|
| Name | Polynucleotide Sequence | NO |
| Anchor Sequence | ACC AGT GAA TAA TTC TTC ACC TTT AGA CAT | 29 |
| | GGT CGT GTT CAA TAT TCA TGT CTC GT | |
| GFP A2 | /5Phos/GGA GAC AGA AAA TTT GIG ACC ATT AAC | 2 |
| | ATC ACC ATC TAA TTC AAC CAA AAT TGG GAC AAC | |
| GFP A3 | /5Phos/AGT AGT ACA AAT AAA TTT TAA GGT CAA TTT | 3 |
| | ACC GTA AGT AGC ATC ACC TTC ACC TTC ACC | |
| GFP A4 Trunc | CCA TAA CCG AAA GTA GTG ACT AAG GTT GGC | 37 |
| | CAT GGA ACT GGC AAT TTA CC | |
| GFP C1 | ATG TCT AAA GGT GAA GAA TTA TTC ACT GGT | 8 |
| | GTT GTC CCA ATT TTG GTT GAA TTA GAT GGT | |
| GFP C2 | /5Phos/GAT GTT AAT GGT CAC AAA TTT TCT GTC | 9 |
| | TCC GGT GAA GGT GAA GGT GAT GCT ACT TAC GGT | |
| GFP C3 | /5Phos/AAA TTG ACC TTA AAA TTT ATT TGT ACT | 10 |
| | ACT GGT AAA TTG CCA GTT CCA TGG CCA ACC TTA | |
| GFP C4 Trunc | /5Phos/GTC ACT ACT TTC GGT TAT GG | 75 |
| F primer | ATG TCT AAA GGT GAA GAA TTA TTC A | 14 |
| R primer | CCA TAA CCG AAA GTA GTG AC | 38 |

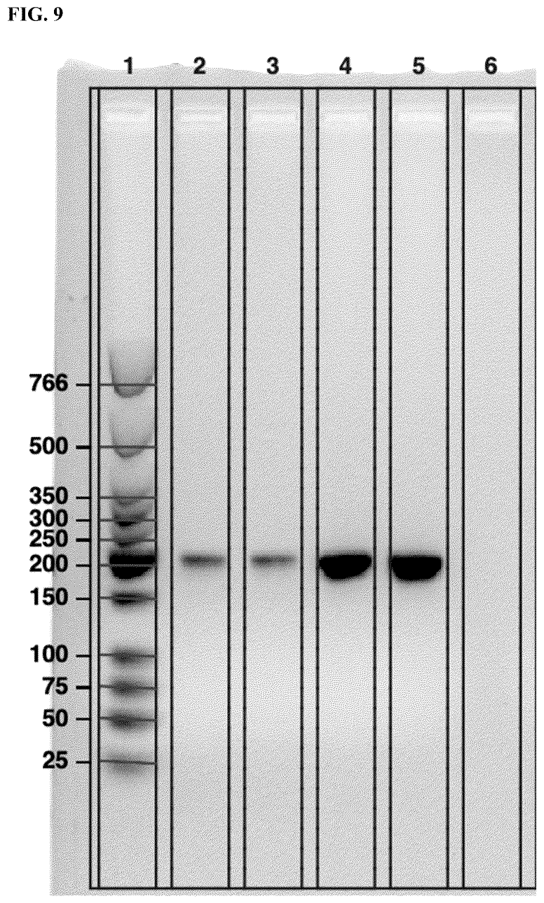

Beads were treated with T4 polynucleotide kinase to add a 5′ phosphate on the anchor sequence to enable downstream ligation. Following phosphorylation, the beads were washed twice with water. To prepare for annealing, 100 picomoles of each polynucleotide for assembly were added to the reaction space in the presence of 2×SSC buffer. Annealing was achieved by increasing the temperature of the reaction to 84° C. and decreasing by 2° C. each minute until the temperature reached 50° C. Several washes were completed with 2×-0.2×SSC with or without 0.1% SDS to remove polynucleotides that were not annealed to the anchored oligo on the glass solid state. Ligation was achieved by adding HiFi™ Taq DNA ligase and the corresponding buffer and incubating for 1 hour at 55° C. Following ligation, the beads were washed with 0.2×SSC with 0.1% SDS and water. The beads were treated with 30 mM NaOH to remove the complementary strand and then washed multiple times with 10 mM Tris-HCl, pH 7.5. A PCR reaction including the primers shown in Table 6, Q5™ polymerase, Q5™ buffer and dNTPs was added to each reaction space. DNA was amplified using 30 PCR cycles. FIG. 9 shows an agarose gel stained with GelGreen™ with the low molecular weight ladder in Lane 1, the PCR products from the silanized beads (no anchor sequence) in Lanes 2 and 3, and the PCR products from the silanized beads with the synthesized anchor sequence in Lanes 4 and 5. A negative water control for PCR is shown in Lane 6.

Surprisingly, non-specifically adsorbed oligonucleotides remained stably attached to the signalized glass beads through multiple washes, enzyme treatments, and temperature changes. The results in this example demonstrate that non-specific adsorption occurs on silanized glass beads during the assembly process.

### Example 6

Another paramagnetic bead type, BcMag™ carboxyl-terminated magnetic beads, was evaluated for specificity and suitability as a solid phase for gene assembly. In this example, the GFP-amine polynucleotide in Table 7 was conjugated to BcMag™ beads by BioClone, Inc.™ and all the beads used in this example were BcMag™ beads with a conjugated the GFP-anchor sequence.

TABLE 7
Polynucleotide sequences used in Example 6

| | | SEQ ID |
|---|---|---|
| Name | Polynucleotide Sequence | NO |
| GFP-amine | /5Phos/ACCAGTGAATAATTCTTCACCTTTAGAC | 2 |
| anchor | ATAATATTTTTTTTTTTT/3AmMC6T/ | |
| GFP A2 | /5Phos/GGA GAC AGA AAA TTT GTG ACC ATT | 2 |
| | AAC ATC ACC ATC TAA TTC AAC CAA AAT | |
| GFP A3 | /5Phos/AGT AGT ACA AAT AAA TTT TAA GGT CAA | 3 |
| | TTT ACC GTA AGT AGC ATC ACC TTC ACC TTC ACC | |
| GFP A4 | /5Phos/ACA TTG AAC ACC ATA ACC GAA AGT | 4 |
| | AGT GAC TAA GGT TGG CCA TGG AAC TGG | |
| GFP A5a | TTG TTT CAT ATG ATC TGG GTA TCT AGC AAA | 39 |
| GFP C1 ext | AAA AAA AAT ATT ATG TCT AAA GGT GAA GAA | 40 |
| | TTA TTC ACT GGT GTT GTC CCA ATT GTT GAA TTA | |
| GFP C2 | /5Phos/GAT GTT AAT GGT CAC AAA TTT TCT GTC | 9 |
| | TCC GGT GAA GGT GAA GGT GAT GCT ACT TAC | |
| GFP C3 | /5Phos/AAA TTG ACC TTA AAA TTT ATT TGT ACT | 10 |
| | ACT GGT AAA TTG CCA GTT CCA TGG CCA ACC | |
| GFP C4 | /5Phos/GTC ACT ACT TTC GGT TAT GGT GTT CAA | 11 |
| | TGT TTT GCT AGA TAC CCA GAT CAT ATG AAA | |
| F GFP | ATG TCT AAA GGT GAA GAA TTA TTC A | 14 |
| primer | | |
| R GFP | TTG TTT CAT ATG ATC TGG GTA TCT A | 41 |
| primer | | |
| Rubisco C1 | AAT ATT ATG GCC AAT TCG AAA ATG GCG ATA | 42 |
| | GCT TCT CTA GCA GCT CAA ATC CGT CCC TTC | |
| Rubisco C2 | /5Phos/TTG GCC GCG TCT CTG CCG TCA CGG TTG | 43 |
| | GCG CCT CAT CCA CCA GAC CTC ATC CGG TGG | |
| Rubisco C3 | /5Phos/AAG CGA GAA GGC GGC TTC GTT CAC CAC | 44 |
| | GCG GTT AAG CTC TCT CAG GAG ACT CAA TTT | |
| Rubisco C4 | /5Phos/ATC GGA CTC ATT TCC ACC GAG CAA ATC | 45 |
| | TCT CCA GGC ACC GAC CTA ATC TCT CTC CCT CCT | |
| Rubisco C5 | /5Phos/CAC GTT CCC TTA CGC TTC GAA TCA GAT | 46 |
| | GAT TCG TCA TCT TCA TCG TCC TCT CTC CTC TCC | |
| Rubisco C6 | /5Phos/GCA TTG GCC CGT CGT GTT CCT GAA GAA | 47 |
| | CTG TGG GCA ATG AAA TTG GGA TTG AGG TTG | |
| Rubisco A2 | /5Phos/CGC CAA CCG TGA CGG CAG AGA CGC GGC | 48 |
| | CAA ACA TGT GAA GGG ACG GAT 'FIG AGC TGC | |
| Rubisco A3 | /5Phos/CGC GTG GTG AAC GAA GCC GCC TTC TCG | 49 |
| | CTT GAT CCA CCG GAT GAG GTC TGG TGG ATG | |
| Rubisco A4 | /5Phos/AGA GAT TTG CTC GGT GGA AAT GAG TCC | 50 |
| | GAT | |
| Rubisco A5 | /5Phos/ATC ATC TGA TTC GAA GCG TAA GGG AAC | 51 |
| | GTG AGG AGG GAG AGA GAT TAG GTC GGT GCC | |
| Rubisco A6 | /5Phos/CAG TTC TTC AGG AAC ACG ACG GGC CAA | 52 |
| | TGC GGA GAG GAG AGA GGA CGA TGA AGA TGA | |
| Rubisco A7 | /5Phos/CCA GAA AGA ATC TGC ATT GGC TCT TTC | 53 |
| | TTG TAA CAA CCT CAA TCC CAA TTT CAT TGC CCA | |
| F Rubisco | ATG GCC AAT TCG AAA ATG G | 54 |
| primer | | |
| R Rubisco | CCA GAA AGA ATC TGC ATT GGC | 55 |
| primer | | |

In this example, two different washing protocols were evaluated with different assembly targets and with different amounts of beads used during the PCR step. All the polynucleotides used for assembly are shown in Table 7 along with the corresponding primers used to amplify each target. The off-target Rubisco assembly was used to evaluate the non-specific adsorption potential of the beads conjugated with the GFP-specific anchor. The Rubisco polynucleotides did not contain a sequence that is complementary to the GFP anchor and therefore should not anneal, ligate, or amplify using the GFP-conjugated beads. It was expected that in the absence of non-specific adsorption, only GFP target should have been assembled and amplified.

Aliquots of beads were washed with Buffer A (10 mM Tris, pH 7.5, 150 mM NaCl, 0.1% BSA) or Buffer B (0.2×SSC, 0.1% BSA). The initial wash of each bead set was done in a final concentration of 0.5% BSA. 100 picomoles of each oligo needed to assemble either Rubisco or GFP (Table 7) were added to beads that were washed with either Buffer A or Buffer B (4 reactions total). Annealing was achieved by increasing the temperature of the reaction to 92° C. and decreasing by 2° C. each minute until the temperature reached 44° C. Each bead sample was washed twice with either Buffer A or Buffer B. Ligation was achieved by adding HiFi™ Taq DNA ligase, buffer, and 0.1% BSA and incubating for 1 hour at 55° C. Following ligation, the beads were washed with Buffer A or Buffer B. Post ligation, aliquots of beads were added to PCR reactions following the schematic in Table 8 below. All PCR reactions contained Q5™ polymerase, Q5™ PCR buffer, dNTPs, 400 nM of each primer, and BSA and were run for 25 cycles. Rubisco primers were used to amplify products assembled with Rubisco polynucleotides and GFP primers were used to amplify products assembled with GFP polynucleotides.

TABLE 8
Sample schematic for Example 6

| | | | Volume of Beads in |
|---|---|---|---|
| Sample Number | Assembly Target | Wash Buffer | PCR (μL) |
| 1 | Rubisco | Buffer A | 1 |
| 2 | Rubisco | Buffer A | 4 |
| 3 | Rubisco | Buffer B | 1 |
| 4 | Rubisco | Buffer B | 4 |
| 5 | GFP | Buffer A | 1 |
| 6 | GFP | Buffer A | 4 |
| 7 | GFP | Buffer B | 1 |
| 8 | GFP | Buffer B | 4 |

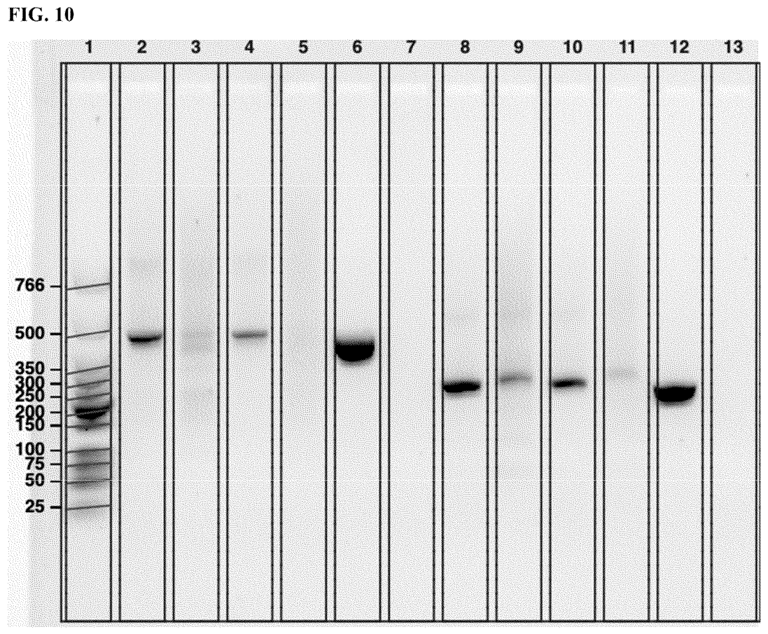

FIG. 10 shows an agarose gel stained with GelGreen™ with low molecular weight ladder in Lane 1 and with Samples 1-4 in Table 8 in Lanes 2-5. Lane 6 contains a PCR positive control for Rubisco and Lane 7 is a negative water control for the Rubisco PCR reaction. Samples 5-8 in bb are in Lanes 8-11 of FIG. 10. Lane 12 contains a positive PCR control for GFP and lane 13 contains a negative PCR control for GFP.

The data in Example 6 show that the GFP-conjugated beads from Bioclone™ exhibited non-specific adsorption of polynucleotides as demonstrated by the amplification of Rubisco from beads that were conjugated with a GFP-amine anchor. The two washing buffers tested in this example were not able to eliminate the non-specific adsorption. Furthermore, these beads also inhibited PCR, which makes them unsuitable for the desired assembly process. As demonstrated when comparing Lanes 8 and 9 to Lanes 10 and 11, less PCR product is observed in the reactions containing a higher bead volume. Because the beads contain the PCR template attached to the surface of the bead, it could be expected that a reaction with a higher bead volume (more template) would result in a higher yield of final PCR product. Surprisingly, increasing the bead volume yielded a lower amount of final PCR product.

### Example 7

Streptavidin-coated PCR tubes were evaluated as a solid state in Example 7 in which full-length GFP was assembled with and without a biotin-labeled universal anchor polynucleotide. The universal anchor polynucleotide was designed so that it would have minimum homology to other potential targets, would not contain hairpins, and would not match common next-generation sequencing adapter sequences. In this example, the anchored strand is attached by 5′-biotinylated end and the complementary strand of the assembly is used as the template during the final PCR step which occurs in liquid phase. The first polynucleotide of the complementary strand of the GFP assembly was designed such that the first 30 nt of the polynucleotide included the complement to the universal anchor sequence and the second 30 nt included the gene-specific sequence for GFP. The remaining polynucleotides were chemically synthesized in 60-nt units with 30-nt overlaps with the complementary polynucleotide on the opposite strand. In this Example, all the steps for solid state assembly of dsDNA were performed (including annealing, ligation, error correction, and PCR) in the same tube, which enabled fast and simple buffer exchange. The 15 polynucleotides used for assembly in this Example are shown in Table 9.

TABLE 9
Polynucleotides used in Example 7

| Polynucleotide | | SEQ ID |
|---|---|---|
| Name | Polynucleotide Sequence | NO |
| UnvAnc | /5Biosg/TTT TTA AGT GCA GGT TAT AAG CGT | 56 |
| 5′ Biotin | CAG GTG GTC CG/3Cy3Sp/ | |
| UnvAnc | TTA TTA AGT GCA GGT TAT AAG CGT CAG GTG GTC | 56 |
| no Biotin | CG/3Cy3Sp/ | |
| GFP A1 | /5Phos/ATG TCT AAA GGT GAA GAA TTA TTC ACT GGT | 57 |
| | GTT GTC CCA ATT TTG GTT GAA TTA GAT GGT | |
| GFP A2 | /5Phos/GGA GAC AGA AAA TTT GTG ACC ATT AAC | 2 |
| | ATC ACC ATC TAA TTC AAC CAA AAT TGG GAC AAC | |
| GFP A3 | /5Phos/AGT AGT ACA AAT AAA TTT TAA GGT CAA TTT | 3 |
| | ACC GTA AGT AGC ATC ACC TTC ACC TTC ACC | |
| GFP A4 | /5Phos/ACA TTG AAC ACC ATA ACC GAA AGT AGT | 4 |
| | GAC TAA GGT TGG CCA TGG AAC TGG CAA TTT ACC | |
| GFP A5 | /5Phos/TTC TGG CAT GGC AGA CTT GAA AAA GTC | 5 |
| | ATG TTG TTT CAT ATG ATC TGG GTA TCT AGC AAA | |
| GFP A6 | /5Phos/AGC TCT GGT CTT GTA GTT ACC GTC ATC TTT | 6 |
| | GAA AAA AAT AGT TCT TTC TTG AAC ATA ACC | |
| GFP A7 | /5Phos/AAA ATC AAT ACC TTT TAA TTC GAT TCTATT | 16 |
| | AAC TAA GGT ATC ACC TTC AAA CTT GAC TTC | |
| GFP A8 | /5Phos/AAC ATT GTG AGA GTT ATA GTT GTA TTC CAA | 17 |
| | TTT GTG ACC TAA AAT GTT ACC ATC TTC TTT | |
| GFP A9 | /5Phos/GTT G'FG TCT AAT TIT GAA GTT AAC TTT GAT | 18 |
| | ACC ATT CTT TTG TTT GTC AGC CAT GAT GTA | |
| GFP A10 | /5Phos/ATC ACC AAT TGG AGT ATT TTG TTG ATA ATG | 19 |
| | GTC AGC TAA TTG AAC AGA ACC ATC TTC AAT | |
| GFP A11 | /5Phos/ATC TTT GGA TAA GGC AGA TTG AGT GGA | 20 |
| | TAA GTA ATG GTT GTC TGG TAA CAA GAC TGG ACC | |
| GFP A12 | /5Phos/GGT AAT ACC AGC AGC AGT AAC AAA TTC | 58 |
| | TAA CAA GAC CAT GTC GTC TCT CTT TTC GTT TGG | |
| GFP A13 | TTA TTT GTA CAA TTC ATC CAT ACC ATG | 28 |
| GFP C1 | /5Phos/ACC AGT GAA TAA TTC TTC ACC TTT AGA CAT | 59 |
| | CGG ACC ACC TGA CGC TTA TAA CCT GCA CTT | |
| GFP C2 | /5Phos/GAT GTT AAT GGT CAC AAA TTT TCT GTC TCC | 9 |
| | GUT GAA GGT GAA GGT GAT GCT ACT TAC GGT | |
| GFP C3 | /5Phos/AAA TTG ACC TTA AAA TTT ATT TGT ACT ACT | 10 |
| | GGT AAA TTG CCA GTT CCA TGG CCA ACC TTA | |
| GFP C4 | /5Phos/GTC ACT ACT TTC GOT TAT GGT GTT CAA TGT | 11 |
| | TTT GCT AGA TAC CCA GAT CAT ATG AAA CAA | |
| GFP C5 | /5Phos/CAT GAC TTT TTC AAG TCT GCC ATG CCA GAA | 12 |
| | GGT TAT GTT CAA GAA AGA ACT ATT TTT TTC | |
| GFP C6 | /5Phos/AAA GAT GAC GGT AAC TAC AAG ACC AGA | 13 |
| | GCT GAA GTC AAG TTT GAA GGT GAT ACC TTA GTT | |
| GFP C7 | /5Phos/AAT AGA ATC GAA TTA AAA GGT ATT GAT TTT | 22 |
| | AAA GAA GAT GGT AAC ATT TTA GGT CAC AAA | |
| GFP C8 | /5Phos/TTG GAA TAC AAC TAT AAC TCT CAC AAT GTT | 23 |
| | TAC ATC ATG GCT GAC AAA CAA AAG AAT GGT | |
| GFP C9 | /5Phos/ATC AAA GTT AAC TTC AAA ATT AGA CAC | 24 |
| | AAC ATT GAA GAT GGT TCT GTT CAA TTA GCT GAC | |
| GFP C10 | /5Phos/CAT TAT CAA CAA AAT ACT CCA ATT GGT GAT | 25 |
| | GGT CCA GTC TTG TTA CCA GAC AAC CAT TAC | |
| GFP C11 | /5Phos/TTA TCC ACT CAA TCT OCC TTA TCC AAA GAT | 26 |
| | CCA AAC GAA AAG AGA GAC CAC ATG GTC TTG | |
| GFP C12 | /5Phos/TTA GAA TTT GTT ACT GCT GCT GGT ATT ACC | 60 |
| | CAT GGT ATG GAT GAA TTG TAC AAA TAA | |
| GFP F primer | ATG TCT AAA GGT GAA GAA TTA TTC A | 14 |
| GFP R primer | TTA TTT GTA CAA TTC ATC CAT ACC ATG | 28 |

To prepare the streptavidin-coated PCR tubes for assembly, universal anchor polynucleotides with or without a 5′ biotin label were added to each tube with 1×BW buffer (5 mM Tris-HCl, pH 7.5, 0.5 mM EDTA, 1 M NaCl) incubated for 30 minutes to facilitate the biotin-streptavidin binding. The tubes incubated with the non-biotinylated polynucleotides were used as a negative control to monitor for non-specific adsorption throughout the assembly process. Following the anchor polynucleotide incubation, the tubes were washed twice with 1×BW to remove excess unbound polynucleotides. One picomole of each of the polynucleotides for assembly listed in Table 9 above was added to the tubes in the presence of 1×BW buffer. Annealing was achieved by increasing the temperature of the reaction to 85° C. and decreasing by 2° C. each minute until the temperature reached 37° C. Following annealing, the unannealed oligos were removed and the tube was washed once with 1×BW buffer. Ligation was achieved by adding HiFi™ Taq DNA ligase and buffer and incubating for 1 hour at 55° C. Following ligation, the ligation reaction buffer and enzyme were removed, the tubes were washed once with 1×BW buffer, and NEB2™ buffer containing either 1 U or 10 U of T7 Endonuclease I™ was added to each tube to correct errors in the dsDNA according to the experiment set-up below in Table 10.

The error correction reaction was incubated for 15 min at 37° C.

TABLE 10
Sample schematic for Example 7

| Sample Number | Anchor Polynucleotide | Units of T7 Endonuclease I |
|---|---|---|
| 1 | Biotin | 1 |
| 2 | Biotin | 1 |
| 3 | Biotin | 10 |
| 4 | Biotin | 10 |
| 5 | No Biotin | 1 |
| 6 | No Biotin | 1 |
| 7 | No Biotin | 10 |
| 8 | No Biotin | 10 |

Following the error correction step, the buffer and enzyme were removed, and the tubes were washed once with 1×BW buffer. PCR reagents including Q5™ polymerase, Q5™ buffer, the primers in Table 9, and dNTPs were added to the tubes, and 20 cycles of thermal cycling were completed.

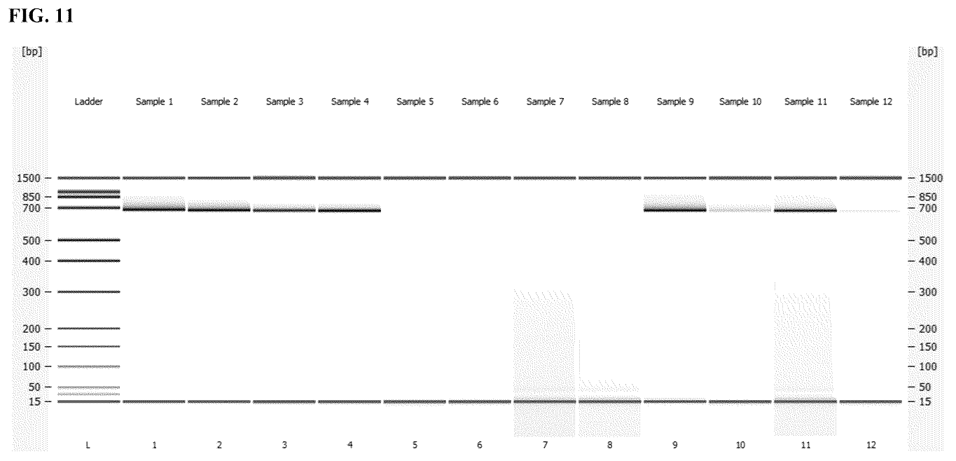

Samples 1-4 were further purified using AIV1Pure™ beads and the manufacturer's recommended protocol. 1 μL of the purified or not purified amplification products were run on an Agilent™ Bioanalyzer™ DNA chip in the following order as shown in FIG. 11.

This Example demonstrated that the streptavidin-coated PCR tubes could be an effective substrate for solid state dsDNA assembly. Only the tubes containing the biotin-labeled polynucleotide amplified a product as shown in FIG. 11, which indicated that this surface had minimal non-specific adsorption. Furthermore, sufficient template DNA remained attached to the solid state to yield amplified product even after several assembly steps and using significantly less input polynucleotide relative to the other examples.

### Example 8

The specificity of the anchor sequences used for assembly on the streptavidin-coated PCR tubes was tested using the universal anchor sequence with the GFP polynucleotide set that included the polynucleotide that is complementary to the universal anchor sequence (GFP Cl, Table 11) and the anchor sequence for Rubisco with the Rubisco polynucleotide set (Rubisco Cl, Table 11).

TABLE 11
Polynucleotides used in Example 8

| Polynucleotide | | SEQ ID |
|---|---|---|
| Name | Polynucleotide Sequence | NO |
| UnvAnc | /5Biosg/TTT TTA AGT GCA GGT TAT AAG CGT | 56 |
| 5′ Biotin | CAG GTG GTC CG/3Cy3Sp/ | |
| GFP A1 | /5Phos/ATG TCT AAA GGT GAA GAA TTA TTC ACT GGT | 57 |
| | GTT GTC CCA ATT TTG GTT GAA TTA GAT GGT | |
| GFP A2 | /5Phos/GGA GAC AGA AAA TTT GTG ACC ATT AAC | 2 |
| | ATC ACC ATC TAA TTC AAC CAA AAT TGG GAC AAC | |
| GFP A3 | /5Phos/AGT AGT ACA AAT AAA TTT TAA GGT CAA TTT | 3 |
| | ACC GTA AGT AGC ATC ACC TTC ACC TTC ACC | |
| GFP A4 | /5Phos/ACA TTG AAC ACC ATA ACC GAA AGT AGT | 4 |
| | GAC TAA GGT TGG CCA TGG AAC TGG CAA TTT ACC | |
| GFP A5 | /5Phos/TTC TGG CAT GGC AGA CTT GAA AAA GTC | 5 |
| | ATG TTG TTT CAT ATG ATC TGG GTA TCT AGC AAA | |
| GFP A6 | /5Phos/AGC TCT GGT CTT GTA GTT ACC GTC ATC TTT | 6 |
| | GAA AAA AAT AGT TCT TTC TTG AAC ATA ACC | |
| GFP A7 | /5Phos/AAA ATC AAT ACC TTT TAA TTC GAT TCTATT | 16 |
| | AAC TAA GGT ATC ACC TTC AAA CTT GAC TTC | |
| GFP A8 | /5Phos/AAC ATT GTG AGA GTT ATA GTT GTA TTC CAA | 17 |
| | TTT GTG ACC TAA AAT GTT ACC ATC TTC TTT | |
| GFP A9 | /5Phos/GTT G′FG TCT AAT TIT GAA GTT AAC TTT GAT | 18 |
| | ACC ATT CTT TTG TTT GTC AGC CAT GAT GTA | |
| GFP A10 | /5Phos/ATC ACC AAT TGG AGT ATT TTG TTG ATA ATG | 19 |
| | GTC AGC TAA TTG AAC AGA ACC ATC TTC AAT | |
| GFP A11 | /5Phos/ATC TTT GGA TAA GGC AGA TTG AGT GGA | 20 |
| | TAA GTA ATG GTT GTC TGG TAA CAA GAC TGG ACC | |
| GFP A12 | /5Phos/GGT AAT ACC AGC AGC AGT AAC AAA TTC | 58 |
| | TAA CAA GAC CAT GTC GTC TCT CTT TTC GTT TGG | |
| GFP A13 | TTA TTT GTA CAA TTC ATC CAT ACC ATG | 28 |
| GFP C1 | /5Phos/ACC AGT GAA TAA TTC TTC ACC TTT AGA CAT | 59 |
| | CGG ACC ACC TGA CGC TTA TAA CCT GCA CTT | |
| GFP C2 | /5Phos/GAT GTT AAT GGT CAC AAA TTT TCT GTC TCC | 9 |
| | GUT GAA GGT GAA GGT GAT GCT ACT TAC GGT | |
| GFP C3 | /5Phos/AAA TTG ACC TTA AAA TTT ATT TGT ACT ACT | 10 |
| | GGT AAA TTG CCA GTT CCA TGG CCA ACC TTA | |
| GFP C4 | /5Phos/GTC ACT ACT TTC GOT TAT GGT GTT CAA TGT | 11 |
| | TTT GCT AGA TAC CCA GAT CAT ATG AAA CAA | |
| GFP C5 | /5Phos/CAT GAC TTT TTC AAG TCT GCC ATG CCA GAA | 12 |
| | GGT TAT GTT CAA GAA AGA ACT ATT TTT TTC | |
| GFP C6 | /5Phos/AAA GAT GAC GGT AAC TAC AAG ACC AGA | 13 |
| | GCT GAA GTC AAG TTT GAA GGT GAT ACC TTA GTT | |
| GFP C7 | /5Phos/AAT AGA ATC GAA TTA AAA GGT ATT GAT TTT | 22 |
| | AAA GAA GAT GGT AAC ATT TTA GGT CAC AAA | |
| GFP C8 | /5Phos/TTG GAA TAC AAC TAT AAC TCT CAC AAT GTT | 23 |
| | TAC ATC ATG GCT GAC AAA CAA AAG AAT GGT | |
| GFP C9 | /5Phos/ATC AAA GTT AAC TTC AAA ATT AGA CAC | 24 |
| | AAC ATT GAA GAT GGT TCT GTT CAA TTA GCT GAC | |
| GFP C10 | /5Phos/CAT TAT CAA CAA AAT ACT CCA ATT GGT GAT | 25 |
| | GGT CCA GTC TTG TTA CCA GAC AAC CAT TAC | |
| GFP C11 | /5Phos/TTA TCC ACT CAA TCT OCC TTA TCC AAA GAT | 26 |
| | CCA AAC GAA AAG AGA GAC CAC ATG GTC TTG | |
| GFP C12 | /5Phos/TTA GAA TTT GTT ACT GCT GCT GGT ATT ACC | 60 |
| | CAT GGT ATG GAT GAA TTG TAC AAA TAA | |
| GFP F primer | ATG TCT AAA GGT GAA GAA TTA TTC A | 14 |
| GFP R primer | TTA TTT GTA CAA TTC ATC CAT ACC ATG | 28 |
| Rubisco Anchor | /5Phos/AGA AGC TAT CGC CAT TTT CHA ATT GGC CAT | 61 |
| | AAT ATT TTT TTT TTT T/3Bio/ | |
| Rubisco C1 | AAT ATT ATG GCC AAT TCG AAA ATG GCG ATA GCT | 42 |
| | TCT CTA GCA GCT CAA ATC CGT CCC TTC ACA TGT | |
| Rubisco C2 | /5Phos/TTG GCC GCG TCT CTG CCG TCA CGG TTG GCG | 43 |
| | CCT CAT CCA CCA GAC CTC ATC CGG TGG ATC | |
| Rubisco C3 | /5Phos/AAG CGA GAA GGC GGC TTC GTT CAC CAC GCG | 44 |
| | GTT AAG CTC TCT CAG GAG ACT CAA TTT GGT | |
| Rubisco C4 | /5Phos/ATC GGA CTC ATT TCC ACC GAG CAA ATC TCT | 45 |
| | CCA GGC ACC GAC CTA ATC TCT CTC CCT CCT | |
| Rubisco C5 | /5Phos/CAC GTT CCC TTA CGC TTC GAA TCA GAT GAT | 46 |
| | TCG TCA TCT TCA TCG TCC TCT CTC CTC TCC | |
| Rubisco C6 | /5Phos/GCA TTG GCC CGT CGT GTT CCT GAA GAA CTG | 47 |
| | TGG GCA ATG AAA TTG GGA TTG AGG TTG TTA | |
| Rubisco C7 | /5Phos/CAA GAA AGA GCC AAT GCA GAT TCT TTC | 63 |
| | TGG TGG CCA TAC ATT AGC AAT CTC CCG GAA ACT | |
| Rubisco C8 | /5Phos/TAT ACT GTT CCC ATA TTC TTT CCC GGA GAA | 64 |
| | GAT ATC AAA AAC TTG CAA TAT GCT CCT CTT | |
| Rubisco C9 | /5Phos/CTC CAC CAG GTC AAC AAA AGA TGC CGT TTT | 65 |
| | CTT CTT GAG TTT GAG CAA GAG ATT AGA CGT | |
| Rubisco C10 | /5Phos/ACT CTT GAA GAT GTT AAG GCT AGT GAT CAT | 66 |
| | CCT TTC AGT GGC CAA GAT GTA AAT GCA TCT | |
| Rubisco C11 | /5Phos/GCC CTA GGG TGG ACT ATG TCA GCT GTT TCA | 67 |
| | ACT AGA GCG TTT CGT TTA CAT GGT AAT AAA | |
| Rubisco C12 | /5Phos/AAG CTT CAA GGT GGA TCT TCT GAT GAT GTT | 68 |
| | CCC ATG ATG CTT CCT TTG ATA GAT ATG TGC | |
| Rubisco A2 | /5Phos/CGC CAA CCG TGA CGG CAG AGA CGC GGC CAA | 48 |
| | ACA TGT GAA GGG ACG GAT ′FIG AGC TGC TAG | |
| Rubisco A3 | /5Phos/CGC GTG GTG AAC GAA GCC GCC TTC TCG CTT | 49 |
| | GAT CCA CCG GAT GAG GTC TGG TGG ATG AGG | |
| Rubisco A4 | /5Phos/AGA GAT TTG CTC GGT GGA AAT GAG TCC GAT | 50 |
| | ACC AAA TTG AGT CTC CTG AGA GAG CTT AAC | |
| Rubisco A5 | /5Phos/ATC ATC TGA TTC GAA GCG TAA GGG AAC GTG | 51 |
| | AGG AGG GAG AGA GAT TAG GTC GGT GCC TGG | |
| Rubisco A6 | /5Phos/CAG TTC TTC AGG AAC ACG ACG GGC CAA TGC | 52 |
| | GGA GAG GAG AGA GGA CGA TGA AGA TGA CGA | |
| Rubisco A7 | /5Phos/CCA GAA AGA ATC TGC ATT GGC TCT TTC TTG | 53 |
| | TAA CAA CCT CAA TCC CAA TTT CAT TGC CCA | |
| Rubisco A8 | /5Phos/TTC TCC GGG AAA GAA TAT GGG AAC AGT | 69 |
| | ATA AGT TTC CGG GAG ATT GCT AAT GTA TGG CCA | |
| Rubisco A9 | /5Phos/AAA ACG GCA TCT TTT GTT GAC CTG GTG GAG | 70 |
| | AAG AGG AGC ATA TTG CAA GTT TTT GAT ATC | |
| Rubisco A10 | /5Phos/ATG ATC ACT AGC CTT AAC ATC TTC AAG AGT | 71 |
| | ACG TCT AAT CTC TTG CTC AAA CTC AAG AAG | |
| Rubisco A11 | /5Phos/ TGA AAC AGC TGA CAT AGT CCA CCC TAG | 72 |
| | GGC AGA TGC ATT TAC ATC TTG GCC ACT GAA AGG | |
| Rubisco A12 | /5Phos/AAC ATC ATC AGA AGA TCC ACC TTG AAG CTT | 73 |
| | TTT ATT ACC ATG TAA ACG AAA CGC TCT AGT | |
| Rubisco A13 | /5Phos/GAG CAT TAG GTT TGA AGC TGT GGT TGC ACA | 74 |
| | TAT CTA TCA AAG GAA GCA TCA TGG G | |
| F Rubisco primer | ATG GCC AAT TCG AAA ATG G | 54 |
| R Rubisco primer | GAG CAT TAG GTT TGA AGC TG | 77 |

The universal anchor sequence (UnvAnc 5′ Biotin) was bound to a set of streptavidin coated tubes and the GFP polynucleotide set or Rubisco polynucleotide set were used in the assembly process with the expectation that only the complementary GFP set would amplify a final product. In parallel, 3′ biotin-labeled gene-specific Rubisco anchor was bound to the streptavidin-coated tubes and the GFP polynucleotide set or Rubisco polynucleotide set were used in the assembly process with the expectation that only the complementary Rubisco set would amplify a final product. The experiment schematic is shown in Table 12.

TABLE 12
Experimental design for Example 8

| Sample Number | Anchor Sequence | Polynucleotide Set | Primer Set |
|---|---|---|---|
| 1 | UnvAnc (GFP comp) | GFP | GFP |
| 2 | UnvAnc (GFP comp) | GFP | GFP |
| 3 | UnvAnc (GFP comp) | Rubisco | Rubisco |
| 4 | UnvAnc (GFP comp) | Rubisco | Rubisco |
| 5 | Rubisco | Rubisco | Rubisco |
| 6 | Rubisco | Rubisco | Rubisco |
| 7 | Rubisco | GFP | GFP |
| 8 | Rubisco | GFP | GFP |

To prepare the streptavidin-coated PCR tubes for assembly, anchor polynucleotides with a biotin label were added to each tube with 1×BW buffer (5 mM Tris-HCl, pH 7.5, 0.5 mM EDTA, 1 M NaCl) and allowed to incubate for 30 minutes to facilitate the biotin-streptavidin binding. Following the anchor polynucleotide incubation, the tubes were washed twice with 1×BW buffer to remove excess unbound polynucleotides. One picomole of each of the polynucleotides for assembly listed in Table 11 and specified by assembly target in Table 12 above was added to the tubes in the presence of 1×BW buffer. Annealing was achieved by increasing the temperature of the reaction to 85° C. and decreasing by 2° C. each minute until the temperature reached 37° C. Following annealing, the unannealed polynucleotides were removed and the tube was washed once with 1×BW buffer. Ligation was achieved by adding HiFi™ Taq DNA ligase and buffer and incubating for 1 hour at 55° C. Following ligation, the ligation reaction buffer and enzyme were removed, the tubes were washed once with 1×BW buffer, and NEB2™ buffer containing 10 U of T7 Endonuclease I™ was added to each tube to correct errors in the dsDNA. The error correction reaction occurred for 15 min at 37° C. Following the error correction step, the buffer and enzyme were removed, and the tubes were washed once with 1×BW buffer. PCR reagents including Q5™ polymerase, Q5™ buffer, the primer sets indicated in Table 12, and dNTPs were added to the tubes, and 20 cycles of thermal cycling were completed.

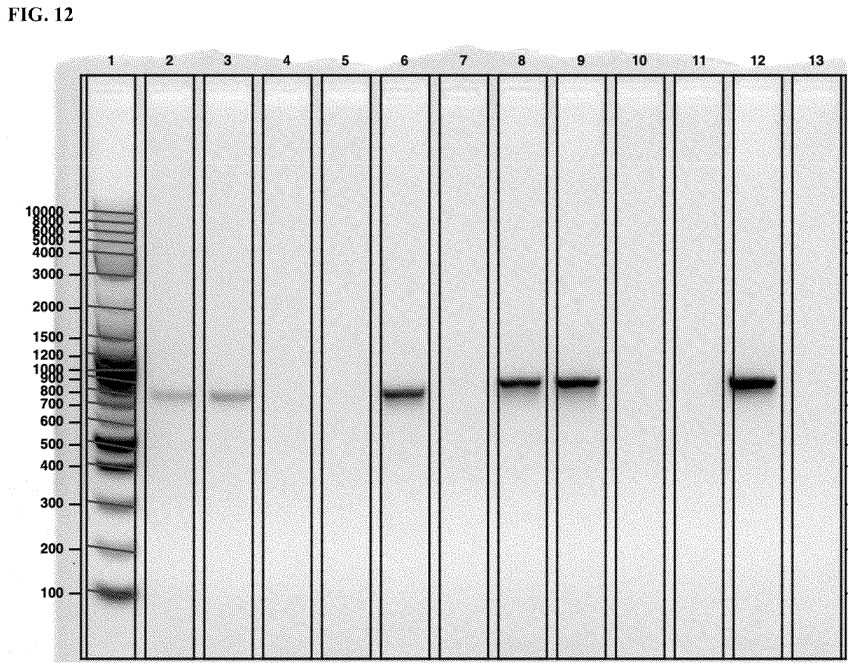

FIG. 12 shows an agarose gel stained with GelGreen™ with the 1 kb Plus™ DNA ladder in Lane 1. The PCR products from Samples 1-4 in Table 12 are shown in Lanes 2-5, with the PCR positive control from the GFP reaction in Lane 6 and the PCR negative control from the GFP reaction in Lane 7. The PCR products from Samples 5-8 in Table 12 are shown in Lanes 8-11, with the positive control for the Rubisco PCR in lane 12 and the negative control for the Rubisco PCR in Lane 13.

These data show that the assembly of genes only occurs in the presence of the corresponding anchor sequence. The assembly method using the streptavidin coated PCR tubes has very low non-specific binding background and has been used to successfully assemble multiple targets.

### Example 9

In this Example, two samples consistent with Example 7 are annealed and ligated. These samples are referred to as samples 9 and 10 The samples are attached to a solid support via a biotin linkage as described in Example 7. Samples 9 and 10 are not treated with T7 Endonuclease I™. In this Example, samples 1-4 from Example 7 and samples 9-10 are sequenced.

It is observed that samples 9 and 10 include the highest number of errors, such as inserted nucleotides, missing nucleotides, and mutated nucleotides. Samples 1 and 2 include some errors, but fewer errors than samples 9 and 10. Samples 3 and 4 include the fewest errors.

From this Example, it is learned that omitting treatment with T7 Endonuclease I™ results in an increased frequency of sequence errors.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

What is claimed is:

1. A method for producing an immobilized double-stranded polynucleotide template, the method comprising:
providing a solid support comprising an attached polynucleotide, wherein the attached polynucleotide is attached to the solid support;
annealing multiple polynucleotides to the attached polynucleotide to yield a double-stranded immobilized polynucleotide assembly comprising annealed polynucleotides, wherein the double-stranded immobilized polynucleotide assembly comprises a top strand with a discontinuous phosphate backbone and a bottom strand with a discontinuous phosphate backbone, wherein the top strand is attached to the solid support;
treating the double-stranded immobilized polynucleotide assembly with a ligase to create a double-stranded immobilized polynucleotide, wherein the multiple polynucleotides of the top strand are ligated to result in the top strand comprising a continuous phosphate backbone and the multiple polynucleotides of the bottom strand are ligated to result in the bottom strand comprising a continuous phosphate backbone; and
treating the double-stranded immobilized polynucleotide with an enzyme to remove any errors in the double-stranded immobilized polynucleotide template.

2. The method of claim 1, wherein the attached polynucleotide is attached to the support by a covalent attachment.

3. The method of claim 1, wherein the attached polynucleotide is attached by 3′ end or the 5′ end.

4. The method of claim 1, wherein one or more of the polynucleotides include chemically synthesized polynucleotides.

5. The method of claim 1, wherein annealing the multiple polynucleotides to the attached polynucleotide comprises annealing at least 3 polynucleotides.

6. The method of claim 1, wherein the annealed polynucleotides include between 20 and 2000 nucleotides.

7. The method of claim 1, wherein treating the double-stranded immobilized polynucleotide assembly with a ligase comprises treating with a Taq-based ligase.

8. The method of claim 1, wherein treating the double-stranded immobilized polynucleotide with an enzyme comprises treating with APE1, endonuclease II, endonuclease IV, endonuclease V, endonuclease VIII, Fpg, hAAG, hSMUG1, mismatch endonuclease I, T4 PDG, T7 endonuclease I, or a combination thereof.

9. The method of claim 1, wherein the error is a mismatched base pair, an abasic site, a site with a nicked backbone, a gap, a chemical error in a nucleotide, a chemical error in a portion of the backbone, or a combination thereof.

10. The method of claim 1, wherein the double-stranded immobilized polynucleotide comprises at least one error, and wherein the method further comprises treating the double-stranded immobilized polynucleotide with an enzyme results in a double-stranded break at the site of the error.

11. The method of claim 1, further comprising denaturing the double-stranded immobilized polynucleotide and removing the bottom strand to yield an immobilized single-stranded polynucleotide template.

12. The method of claim 1, further comprising:
amplifying the immobilized double-stranded polynucleotide template of claim 1 enzymatically with two primers, to result in an amplified template,
wherein amplifying comprises multiple rounds of amplification,
treating the amplified template with 3′-5′ exonuclease; and
separating the amplified template from reaction components to yield an amplified double-stranded polynucleotide.

13. The method of claim 12, wherein the amplified double-stranded polynucleotide has a length of at least 50 nucleotides, at least 100 nucleotides, at least 200 nucleotides, or at least 5,000 nucleotides.

14. The method of claim 12, wherein the amplified double-stranded polynucleotide has a length of at most 2,000 nucleotides, at most 5,000 nucleotides, or at most 10,000 nucleotides.

15. The method of claim 12, wherein amplifying the immobilized polynucleotide template comprises a single amplification cycle.

16. The method of claim 12, wherein amplifying the immobilized polynucleotide template comprises more than one amplification cycle.

17. The method of claim 12, wherein amplifying the immobilized polynucleotide template comprises single-direction amplification.

18. The method of claim 12, wherein the amplified double-stranded polynucleotide includes fewer errors than a double-stranded polynucleotide produced using a comparable method not including treatment with an enzyme to remove an error.

19. A solid support comprising the immobilized polynucleotide templates of claim 1.

20. A kit comprising the solid support of claim 19.


SEQUENCE LISTING (77 sequences)

SEQ ID NO: 1 - DNA, 46 bp, synthetic construct
  source 1..46: mol_type = other DNA
accagtgaataattcttcacctttagacataatatttttttttttt

SEQ ID NO: 2 - DNA, 60 bp, synthetic construct
  source 1..60: mol_type = other DNA
ggagacagaaaatttgtgaccattaacatcaccatctaattcaaccaaaattgggacaac

SEQ ID NO: 3 - DNA, 60 bp, synthetic construct
  source 1..60: mol_type = other DNA
agtagtacaaataaattttaaggtcaatttaccgtaagtagcatcaccttcaccttcacc

SEQ ID NO: 4 - DNA, 60 bp, synthetic construct
  source 1..60: mol_type = other DNA
acattgaacaccataaccgaaagtagtgactaaggttggccatggaactggcaatttacc

SEQ ID NO: 5 - DNA, 60 bp, synthetic construct
  source 1..60: mol_type = other DNA
ttctggcatggcagacttgaaaaagtcatgttgtttcatatgatctgggtatctagcaaa

SEQ ID NO: 6 - DNA, 60 bp, synthetic construct
  source 1..60: mol_type = other DNA
agctctggtcttgtagttaccgtcatctttgaaaaaaatagttctttcttgaacataacc

SEQ ID NO: 7 - DNA, 30 bp, synthetic construct
  source 1..30: mol_type = other DNA
aactaaggtatcaccttcaaacttgacttc

SEQ ID NO: 8 - DNA, 66 bp, synthetic construct
  source 1..66: mol_type = other DNA
aatattatgtctaaaggtgaagaattattcactggtgttgtcccaattttggttgaattagatggt

SEQ ID NO: 9 - DNA, 60 bp, synthetic construct
  source 1..60: mol_type = other DNA
gatgttaatggtcacaaattttctgtctccggtgaaggtgaaggtgatgctacttacggt

SEQ ID NO: 10 - DNA, 60 bp, synthetic construct
  source 1..60: mol_type = other DNA
aaattgaccttaaaatttatttgtactactggtaaattgccagttccatggccaacctta

SEQ ID NO: 11 - DNA, 60 bp, synthetic construct
  source 1..60: mol_type = other DNA
gtcactactttcggttatggtgttcaatgttttgctagatacccagatcatatgaaacaa

SEQ ID NO: 12 - DNA, 60 bp, synthetic construct
  source 1..60: mol_type = other DNA
catgactttttcaagtctgccatgccagaaggttatgttcaagaaagaactatttttttc

SEQ ID NO: 13 - DNA, 60 bp, synthetic construct
  source 1..60: mol_type = other DNA
aaagatgacggtaactacaagaccagagctgaagtcaagtttgaaggtgataccttagtt

SEQ ID NO: 14 - DNA, 25 bp, synthetic construct
  source 1..25: mol_type = other DNA
atgtctaaaggtgaagaattattca

SEQ ID NO: 15 - DNA, 44 bp, synthetic construct
  source 1..44: mol_type = other DNA
catggtatggatgaattgtacaaataaaatattttttttttttt

SEQ ID NO: 16 - DNA, 60 bp, synthetic construct
  source 1..60: mol_type = other DNA
aaaatcaataccttttaattcgattctattaactaaggtatcaccttcaaacttgacttc

SEQ ID NO: 17 - DNA, 60 bp, synthetic construct
  source 1..60: mol_type = other DNA
aacattgtgagagttatagttgtattccaatttgtgacctaaaatgttaccatcttcttt

SEQ ID NO: 18 - DNA, 60 bp, synthetic construct
  source 1..60: mol_type = other DNA
gttgtgtctaattttgaagttaactttgataccattcttttgtttgtcagccatgatgta

SEQ ID NO: 19 - DNA, 60 bp, synthetic construct
  source 1..60: mol_type = other DNA
atcaccaattggagtattttgttgataatggtcagctaattgaacagaaccatcttcaat

SEQ ID NO: 20 - DNA, 60 bp, synthetic construct
  source 1..60: mol_type = other DNA
atctttggataaggcagattgagtggataagtaatggttgtctggtaacaagactggacc

SEQ ID NO: 21 - DNA, 87 bp, synthetic construct
  source 1..87: mol_type = other DNA
ttatttgtacaattcatccataccatgggtaataccagcagcagtaacaaattctaacaagaccatgtggtctctcttttcgtttgg

SEQ ID NO: 22 - DNA, 60 bp, synthetic construct
  source 1..60: mol_type = other DNA
aatagaatcgaattaaaaggtattgattttaaagaagatggtaacattttaggtcacaaa

SEQ ID NO: 23 - DNA, 60 bp, synthetic construct
  source 1..60: mol_type = other DNA
ttggaatacaactataactctcacaatgtttacatcatggctgacaaacaaaagaatggt

SEQ ID NO: 24 - DNA, 60 bp, synthetic construct
  source 1..60: mol_type = other DNA
atcaaagttaacttcaaaattagacacaacattgaagatggttctgttcaattagctgac

SEQ ID NO: 25 - DNA, 60 bp, synthetic construct
  source 1..60: mol_type = other DNA
cattatcaacaaaatactccaattggtgatggtccagtcttgttaccagacaaccattac

SEQ ID NO: 26 - DNA, 60 bp, synthetic construct
  source 1..60: mol_type = other DNA
ttatccactcaatctgccttatccaaagatccaaacgaaaagagagaccacatggtcttg

SEQ ID NO: 27 - DNA, 30 bp, synthetic construct
  source 1..30: mol_type = other DNA
ttagaatttgttactgctgctggtattacc

SEQ ID NO: 28 - DNA, 27 bp, synthetic construct
  source 1..27: mol_type = other DNA
ttatttgtacaattcatccataccatg

SEQ ID NO: 29 - DNA, 56 bp, synthetic construct
  source 1..56: mol_type = other DNA
accagtgaataattcttcacctttagacatggtcgtgttcaatattcatgtctcgt

SEQ ID NO: 30 - DNA, 39 bp, synthetic construct
  source 1..39: mol_type = other DNA
tgaagaattattcactggtgttgtcccaattttggttga

SEQ ID NO: 31 - DNA, 39 bp, synthetic construct
  source 1..39: mol_type = other DNA
gtcacaaattttctgtctccggtgaaggtgaaggtgatg

SEQ ID NO: 32 - DNA, 40 bp, synthetic construct
  source 1..40: mol_type = other DNA
taaaatttatttgtactactggtaaattgccagttccatg

SEQ ID NO: 33 - DNA, 40 bp, synthetic construct
  source 1..40: mol_type = other DNA
tcggttatggtgttcaatgttttgctagatacccagatca

SEQ ID NO: 34 - DNA, 21 bp, synthetic construct
  source 1..21: mol_type = other DNA
acgagacatgaatattgaaca

SEQ ID NO: 35 - DNA, 20 bp, synthetic construct
  source 1..20: mol_type = other DNA
gacttgaaaaagtcatgttg

SEQ ID NO: 36 - DNA, 50 bp, synthetic construct
  source 1..50: mol_type = other DNA
ccataaccgaaagtagtgactaaggttggccatggaactggcaatttacc

SEQ ID NO: 37 - DNA, 20 bp, synthetic construct
  source 1..20: mol_type = other DNA
gtcactactttcggttatgg

SEQ ID NO: 38 - DNA, 20 bp, synthetic construct
  source 1..20: mol_type = other DNA
ccataaccgaaagtagtgac

SEQ ID NO: 39 - DNA, 30 bp, synthetic construct
  source 1..30: mol_type = other DNA
ttgtttcatatgatctgggtatctagcaaa

SEQ ID NO: 40 - DNA, 72 bp, synthetic construct
  source 1..72: mol_type = other DNA
aaaaaaaatattatgtctaaaggtgaagaattattcactggtgttgtcccaattttggttgaattagatggt

SEQ ID NO: 41 - DNA, 25 bp, synthetic construct
  source 1..25: mol_type = other DNA
ttgtttcatatgatctgggtatcta

SEQ ID NO: 42 - DNA, 66 bp, synthetic construct
  source 1..66: mol_type = other DNA
aatattatggccaattcgaaaatggcgatagcttctctagcagctcaaatccgtcccttcacatgt

SEQ ID NO: 43 - DNA, 60 bp, synthetic construct
  source 1..60: mol_type = other DNA
ttggccgcgtctctgccgtcacggttggcgcctcatccaccagacctcatccggtggatc

SEQ ID NO: 44 - DNA, 60 bp, synthetic construct
  source 1..60: mol_type = other DNA
aagcgagaaggcggcttcgttcaccacgcggttaagctctctcaggagactcaatttggt

SEQ ID NO: 45 - DNA, 60 bp, synthetic construct
  source 1..60: mol_type = other DNA
atcggactcatttccaccgagcaaatctctccaggcaccgacctaatctctctccctcct

SEQ ID NO: 46 - DNA, 60 bp, synthetic construct
  source 1..60: mol_type = other DNA
cacgttcccttacgcttcgaatcagatgattcgtcatcttcatcgtcctctctcctctcc

SEQ ID NO: 47 - DNA, 60 bp, synthetic construct
  source 1..60: mol_type = other DNA
gcattggcccgtcgtgttcctgaagaactgtgggcaatgaaattgggattgaggttgtta

SEQ ID NO: 48 - DNA, 60 bp, synthetic construct
  source 1..60: mol_type = other DNA
cgccaaccgtgacggcagagacgcggccaaacatgtgaagggacggatttgagctgctag

SEQ ID NO: 49 - DNA, 60 bp, synthetic construct
  source 1..60: mol_type = other DNA
cgcgtggtgaacgaagccgccttctcgcttgatccaccggatgaggtctggtggatgagg

SEQ ID NO: 50 - DNA, 60 bp, synthetic construct
  source 1..60: mol_type = other DNA
agagatttgctcggtggaaatgagtccgataccaaattgagtctcctgagagagcttaac

SEQ ID NO: 51 - DNA, 60 bp, synthetic construct
  source 1..60: mol_type = other DNA
atcatctgattcgaagcgtaagggaacgtgaggagggagagagattaggtcggtgcctgg

SEQ ID NO: 52 - DNA, 60 bp, synthetic construct
  source 1..60: mol_type = other DNA
cagttcttcaggaacacgacgggccaatgcggagaggagagaggacgatgaagatgacga

SEQ ID NO: 53 - DNA, 60 bp, synthetic construct
  source 1..60: mol_type = other DNA
ccagaaagaatctgcattggctctttcttgtaacaacctcaatcccaatttcattgccca

SEQ ID NO: 54 - DNA, 19 bp, synthetic construct
  source 1..19: mol_type = other DNA
atggccaattcgaaaatgg

SEQ ID NO: 55 - DNA, 21 bp, synthetic construct
  source 1..21: mol_type = other DNA
ccagaaagaatctgcattggc

SEQ ID NO: 56 - DNA, 35 bp, synthetic construct
  source 1..35: mol_type = other DNA
tttttaagtgcaggttataagcgtcaggtggtccg

SEQ ID NO: 57 - DNA, 60 bp, synthetic construct
  source 1..60: mol_type = other DNA
atgtctaaaggtgaagaattattcactggtgttgtcccaattttggttgaattagatggt

SEQ ID NO: 58 - DNA, 60 bp, synthetic construct
  source 1..60: mol_type = other DNA
ggtaataccagcagcagtaacaaattctaacaagaccatgtggtctctcttttcgtttgg

SEQ ID NO: 59 - DNA, 60 bp, synthetic construct
  source 1..60: mol_type = other DNA
accagtgaataattcttcacctttagacatcggaccacctgacgcttataacctgcactt

SEQ ID NO: 60 - DNA, 57 bp, synthetic construct
  source 1..57: mol_type = other DNA
ttagaatttgttactgctgctggtattacccatggtatggatgaattgtacaaataa

SEQ ID NO: 61 - DNA, 46 bp, synthetic construct
  source 1..46: mol_type = other DNA
agaagctatcgccattttcgaattggccataatatttttttttttt

SEQ ID NO: 62 - skipped

SEQ ID NO: 63 - DNA, 60 bp, synthetic construct
  source 1..60: mol_type = other DNA
caagaaagagccaatgcagattctttctggtggccatacattagcaatctcccggaaact

SEQ ID NO: 64 - DNA, 60 bp, synthetic construct
  source 1..60: mol_type = other DNA
tatactgttcccatattctttcccggagaagatatcaaaaacttgcaatatgctcctctt

SEQ ID NO: 65 - DNA, 60 bp, synthetic construct
  source 1..60: mol_type = other DNA
ctccaccaggtcaacaaaagatgccgttttcttcttgagtttgagcaagagattagacgt

SEQ ID NO: 66 - DNA, 60 bp, synthetic construct
  source 1..60: mol_type = other DNA
actcttgaagatgttaaggctagtgatcatcctttcagtggccaagatgtaaatgcatct

SEQ ID NO: 67 - DNA, 60 bp, synthetic construct
  source 1..60: mol_type = other DNA
gccctagggtggactatgtcagctgtttcaactagagcgtttcgtttacatggtaataaa

SEQ ID NO: 68 - DNA, 60 bp, synthetic construct
  source 1..60: mol_type = other DNA
aagcttcaaggtggatcttctgatgatgttcccatgatgcttcctttgatagatatgtgc

SEQ ID NO: 69 - DNA, 60 bp, synthetic construct
  source 1..60: mol_type = other DNA
ttctccgggaaagaatatgggaacagtataagtttccgggagattgctaatgtatggcca

SEQ ID NO: 70 - DNA, 60 bp, synthetic construct
  source 1..60: mol_type = other DNA
aaaacggcatcttttgttgacctggtggagaagaggagcatattgcaagtttttgatatc

SEQ ID NO: 71 - DNA, 60 bp, synthetic construct
  source 1..60: mol_type = other DNA
atgatcactagccttaacatcttcaagagtacgtctaatctcttgctcaaactcaagaag

SEQ ID NO: 72 - DNA, 60 bp, synthetic construct
  source 1..60: mol_type = other DNA
tgaaacagctgacatagtccaccctagggcagatgcatttacatcttggccactgaaagg

SEQ ID NO: 73 - DNA, 60 bp, synthetic construct
  source 1..60: mol_type = other DNA
aacatcatcagaagatccaccttgaagctttttattaccatgtaaacgaaacgctctagt

SEQ ID NO: 74 - DNA, 55 bp, synthetic construct
  source 1..55: mol_type = other DNA
gagcattaggtttgaagctgtggttgcacatatctatcaaaggaagcatcatggg

SEQ ID NO: 75 - DNA, 20 bp, synthetic construct
  source 1..20: mol_type = other DNA
gtcactactttcggttatgg

SEQ ID NO: 76 - DNA, 46 bp, synthetic construct; identical to SEQ ID NO: 1
  source 1..46: mol_type = other DNA

SEQ ID NO: 77 - DNA, 20 bp, synthetic construct
  source 1..20: mol_type = other DNA
gagcattaggtttgaagctg